SEQUENCE LISTING (13 sequences)

SEQ ID NO: 1 - protein, 5 aa, Artificial Sequence
  source: /note=“Description of Artificial Sequence Synthetic peptide”
SYVMH

SEQ ID NO: 2 - protein, 6 aa, Artificial Sequence
  source: /note=“Description of Artificial Sequence Synthetic peptide”
NPYNDG

SEQ ID NO: 3 - protein, 12 aa, Artificial Sequence
  source: /note=“Description of Artificial Sequence Synthetic peptide”
GTYYYGTRVFDY

SEQ ID NO: 4 - protein, 16 aa, Artificial Sequence
  source: /note=“Description of Artificial Sequence Synthetic peptide”
RSSKSLQNVNGNTYLY

SEQ ID NO: 5 - protein, 7 aa, Artificial Sequence
  source: /note=“Description of Artificial Sequence Synthetic peptide”
RMSNLNS

SEQ ID NO: 6 - protein, 9 aa, Artificial Sequence
  source: /note=“Description of Artificial Sequence Synthetic peptide”
MQHLEYPIT

SEQ ID NO: 7 - protein, 121 aa, Artificial Sequence
  source: /note=“Description of Artificial Sequence Synthetic polypeptide”
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 8 - protein, 112 aa, Artificial Sequence
  source: /note=“Description of Artificial Sequence Synthetic polypeptide”
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 9 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 107 aa, Artificial Sequence [sequence omitted]

SEQ ID NO: 11 - protein, 451 aa, Artificial Sequence
  source: /note=“Description of Artificial Sequence Synthetic polypeptide”
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 219 aa, Artificial Sequence
  source: /note=“Description of Artificial Sequence Synthetic polypeptide”
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 556 aa, Homo sapiens [sequence omitted]

(19) United States
(12) Patent Application Publication
(10) Pub. No.: US 2024/0156863 A1
(43) Pub. Date: May 16, 2024

(54) SEQUENTIAL ANTI-CD19 THERAPY

(71) Applicant: MORPHOSYS AG, Planegg (DE)

(72) Inventors: Jan ENDELL, Munich (DE); Stefanie FRICK, Munich (DE)

(21) Appl. No.: 17/773,117

(22) PCT Filed: Oct. 30, 2020
(86) PCT No.: PCT/EP2020/080502

§ 371 Date: Apr. 29, 2022

(30) Foreign Application Priority Data
Oct. 31, 2019 (EP) 19206488.9

(51) Int. Cl. A61K 35/17; A61K 31/454; A61K 39/00; A61P 35/02; C07K 16/28

(52) U.S. Cl. CPC A61K 35/17; A61K 31/454; A61K 39/4611; A61P 35/02; C07K 16/2803; A61K 2039/505; A61K 2239/13

(57) ABSTRACT

The present invention relates to therapeutic compositions and methods for sequential treatment of cancer in human patients using therapeutic agents that bind to human CD1g.

### FIELD OF THE INVENTION

The present invention is directed to immunotherapeutic compositions and methods for sequential treatment of cancer in human patients using therapeutic agents that bind to human CD19. In particular, immunotherapeutic regimens such as CAR-T cells are described, useful for the therapy of B cell malignancies, such as non-Hodgkin's lymphoma (NHL), including diffuse large B cell lymphoma (DLBCL), as well as chronic lymphocytic leukemia (CLL) in patients after prior treatment with the anti-CD19 antibody tafasitamab.

### BACKGROUND OF THE INVENTION

CD19, a co-receptor of the B-cell receptor, is a marker of the B-cell lineage as it is expressed throughout B-cell development until terminal differentiation of B cells into plasma cells (Wang, Exp Hematol Oncol. 2012). CD19 is present on the surface of both healthy and malignant B cells. Most B cell tumors express CD19, examples include acute lymphoblastic leukemia (ALL), chronic lymphocytic leukemia (CLL) and B cell lymphoma. Approximately 90% of diffuse large B-cell lymphomas (DLBCL) express the CD19 antigen (Kimura et al International Journal of Hematology 2007) and about 80% of the ALL cases. The fact that some tumors do not express CD19 indicates that CD19 is not critical for B-cell survival. This is supported by data from CD19 knockout mice that revealed that CD19 deficiency has no effect on the number of early B cell precursor in the bone marrow, nor on the size and morphology of B cells. CD19−/− mice instead show reductions in the total number and frequency of peripheral B cells. Thus, CD19 contributes to the balance between humoral, antigen-induced response and tolerance induction and therefore plays a vital role in mounting an optimal immune response (Wang, Exp Hematol Oncol. 2012).

Because of its ubiquity on malignant B cells, CD19 is a suitable target for immunotherapies. CD19 expression is restricted to B lineage cells and it is not found on pluripotent blood stem cells or on most other normal tissues (Scheuermann, Leuk Lymphoma. 1995). The major drug classes contributing to the CD19 targeting therapeutic market include monoclonal antibodies (e.g. MOR208, tafasitamab; MEDI-551, inebilizumab), antibody-drug conjugates (e.g. SAR3419, coltuximab ravtansine), bispecifics (e.g. blinatumomab, BLINCYTO®) or chimeric antigen receptor T cell (CAR-T) approaches (e.g. axicabtagen-ciloleucel, YESCARTA®; tisagenlecleucel, KYMRIAH®).

Tafasitamab, is an Fc-enhanced, humanized, monoclonal antibody, that targets CD19 and which has been shown to exert its antitumor effect through antibody-dependent cellular toxicity (ADCC), antibody-dependent cellular phagocytosis (ADCP) and direct cytotoxicity (Awan F T et al. Blood. 2010 Feb 11; 115(6):1204-13) (W02008022152). It recently received FDA breakthrough therapy designation. Currently, tafasitamab is being studied in phase II and phase III clinical trials for diffuse large B-cell lymphoma (DLBCL) in combination with the immunomodulatory agent lenalidomide (L-MIND) and the chemotherapeutic drug bendamustine (B-MIND). L-MIND (NCT02399085) is an open-label, single-arm, phase II study of tafasitamab (TAFA) plus lenalidomide (LEN) in patients with relapsed/refractory (R/R) DLBCL ineligible for autologous stem cell transplantation (ASCT). B-MIND (NCT02763319) is an open-label, two-arm, phase II/III efficacy and safety study of tafasitamab in combination with bendamustine (BEN) versus rituximab in patients with (R/R) DLBCL ineligible for high-hose chemotherapy (HDC) and ASCT.

Adoptive cell transfer utilizing autologous T cells genetically engineered ex vivo to target tumor antigens is a promising therapeutic approach for the treatment of CD19 positive hematological malignancies. CD19 directed chimeric antigen receptor T cell (CAR-T) therapy has shown remarkable activity in B cell lymphoma and acute lymphoblastic leukemia and two anti-CD19 CAR-T therapies were approved by the FDA in 2017. These anti-CD19 CAR-T cells have demonstrated significant efficacy in the treatment of patients with relapsed, refractory B cell lymphoid malignancies (Maude S et al, N Engl J Med. 2018, 378:439-48), such as patients with R/R DLBCL after failure on ≥2 lines of systemic therapy (Neelapu S et al, N Engl J Med. 2017, 377:2531-44). Although these studies demonstrated unprecedented efficacy, it also became apparent that not all patients respond to anti-CD19 CAR-T cells, and even for those who initially respond, durability of response remains a limitation. The patients' capacity to respond to CAR-T cell therapy may be influenced by, for example, number or type of previous lines of treatment, elevated expression of inhibitory signals around CAR-T cells (e.g. PD-L1, which cause suppression), changes in target antigen expression or epitope loss.

With the emergence of other CD19 directed modalities for the treatment of B cell malignancies such as monoclonal antibodies and bispecific T cell engagers (BiTe), the question of adequate therapeutic sequencing remains to be addressed. As such, it is unclear whether targeting CD19 with a monoclonal antibody such as tafasitamab would interfere with the ability of CD19 targeting CAR-Ts to exert their anti-tumor effect in a subsequent therapy.

The high potential of immunotherapies in general comes with the caveat of tumor escape, wherein clonal tumor cells develop a mechanism by which they can resist specific therapies. Amongst the earliest identified resistance mechanisms was the downregulation of CD19 from the surface of tumor cells (Grupp S A et al, N Engl J Med. (2013) 368:1509-18, Ruella M Comput Struct Biotechnol J. (2016) 14:357-62). Therapeutic compounds place selective pressure on the tumor and malignant clones that have developed resistance to the therapeutic compound are able to proliferate. For example, in about 10% to 20% patients with acute lymphoblastic leukemia (ALL) under CD19/CD3 bispecific T-cell engager (blinatumomab) treatment, CD19 negative (CD19−) relapses occur. Such immune escape may be facilitated by multiple mechanisms such as lineage switching, immunoediting, epitope loss, splice or exon variants, and point mutations, including the acquisition of secondary CD19 mutations that render CD19 non-functional. Immune escape is a major form of therapeutic resistance for patients with ALL (Braig F et al., 2017 Blood. 129(1):100-104; Grupp S A et al. N Engl J Med. 2013 Apr 18; 368(16):1509-1518). Targeting of specific antigens on a tumor cell surface by monoclonal antibodies can select for clones that are not recognized by the antibody or are not affected by its binding.

In such cases an important question to address in the era of targeted immunotherapy is: can the same tumor antigen be targeted with a different cancer immune therapy modality after disease progression following a previous therapy against that same antigen? In such a clinical scenario, there is concern for sustained antigen blockade from the prior line of therapy. In addition, there is concern for the selective pressures of prior therapy targeted against a specific antigen allowing for progression of a clone that does not express that antigen, rendering subsequent therapy against the same target ineffective (antigen escape). The development of new approaches for attenuating, preventing or obviating antigen-loss escape or without any interference with a previous line of therapy directed against the same antigen would therefore represent an advance in the field.

It is known from the prior art that CD19 may be readily internalized upon Ab binding (Pulczynski S Blood. 1993, 81(6):1549-57) and that loss of CD19 expression on tumor cells is a frequent escape mechanism in patients treated with CD19-targeted T cells (Grupp S A, N Engl J Med. 2013 Apr 18; 368(16):1509-1518). Dual CD19 and CD123 targeting has been described to prevent antigen-loss relapses after CD19-directed immunotherapy (Ruella et al. J Clin Invest. 2016 Oct 3; 126(10): 3814-3826) but this concept uses a combination of multiple targets.

The invention is based on the surprising finding that CD19 can still be targeted after treatment with the monoclonal anti-CD19 antibody tafasitamab (MOR208). It is therefore possible to administer another, optionally different, anti-CD19 moiety after tafasitamab treatment. In particular, the inventors of the present disclosure surprisingly found that there is no functional interference between the tafasitamab and CD19 directed CAR-T cells (CART19). This finding is supported by an unpublished case study showing sustained remission in a patient with relapsed and refractory DLBCL achieved by anti-CD19 CAR-T cell therapy after prior treatment with tafasitamab as part of the L-MIND trial.

### SUMMARY OF THE INVENTION

Disclosed are methods and compositions for treating hematological cancer that comprise administering a composition to a patient comprising autologous T cells, which express a recombinant receptor that specifically binds to an antigen specific for the cancer, wherein the patient was previously treated with a composition comprising an anti-CD19 antibody, the antibody comprising
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab.

More specifically, one embodiment is related to methods and compounds for treating CD19+ hematological cancer. In particular, the antigen specific for the cancer is human CD19. Preferably, the CD19+ hematological cancer is DLBCL. In another preferred embodiment, the DLBCL is relapsed or refractory (R/R) DLBCL.

In one embodiment, the autologous T cells expressing a recombinant receptor are CAR-T cells. In one embodiment, the CAR-T cells are directed to CD20 or CD19. In a preferred embodiment, the CAR-T cells are directed to CD19. More specifically, the CAR-T cells are directed against CD19 and are selected from the group of axicabtagen-ciloleucel (YESCARTA®), tisagenlecleucel (KYMRIAH®), lisocabtagene maraleucel (JCAR017), and UCART19.

In particular, CAR-T cells directed against CD19 are disclosed for use in the treatment of cancer in a patient, wherein the patient was treated previously with an anti-CD19 antibody.

More specifically, one embodiment relates to CAR-T cells directed against CD19 for use in the treatment of cancer in a patient, wherein the patient was treated previously with a composition comprising tafasitamab. In a particular embodiment, tisagenlecleucel CAR-T cells are disclosed for use in the treatment of cancer in a patient, wherein the patient was treated previously with a composition comprising tafasitamab. A particular embodiment relates to CAR-T cells directed against CD19 for use in the treatment of R/R DLBCL in a patient, wherein the patient was treated previously with a composition comprising tafasitamab. Another aspect relates to CAR-T cells directed against CD19 for use in the treatment of R/R DLBCL in a patient, wherein the patient was treated previously with a combination therapy comprising tafasitamab and lenalidomide. In a certain embodiment, tisagenlecleucel CAR-T cells are disclosed for use in the treatment of R/R DLBCL in a patient, wherein the patient was treated previously with a composition comprising tafasitamab. In another aspect, tisagenlecleucel CAR-T cells are disclosed for use in the treatment of R/R DLBCL in a patient, wherein the patient was treated previously with a combination therapy comprising tafasitamab and lenalidomide.

### BRIEF DESCRIPTION OF THE FIGURES

FIG. 1: Cytotoxicity of tafasitamab against Jeko-1 cell line (A) with and without natural killer (NK) cells. Cytotoxicity was assessed 24 hours after incubation of the CD19+, luciferase+Jeko cells with different concentrations of tafasitamab, in the presence or absence of NK cells. In the presence of NK cells tafasitamab demonstrates significantly higher anti-tumor cytotoxicity. Activity was determined by bioluminescence imaging after 24 hours. (n=3 independent experiments, data was consistent with targeting the Nalm-6 and Ly7 cell lines (B and C).

FIG. 2: Clone FMC63 (CD19 binding domain in CART19) and tafasitamab compete for CD19 binding. After 4 hours of treatment of Jeko cells with 2 or 0.5 mg/mL of tafasitamab, flow cytometry using the FMC63 anti-CD19 antibody was performed. A strongly reduced CD19 signal was observed by flow cytometry, indicating a direct competition for binding of tafasitamab and FMC63.

FIG. 3: CART19 exhibit potent antigen specific cytotoxicity in the presence of the monoclonal anti-CD19 antibody tafasitamab. Luciferase positive, CD19+ Jeko cells (A) were incubated with different concentrations of tafasitamab, and then CART19 cells were added 3 hours later at different E:T ratios as indicated. Activity was determined by bioluminescence imaging after 24 hours (n=3 independent experiments) Consistent results were obtained when the Ly7 or Nalm-6 cell lines were used (B and C).

FIG. 4: CART19 cells continue to show significant antigen specific degranulation and cytokine production in the presence of the anti-CD19 antibody tafasitamab. When CART19 cells or untransduced T cells (UTD) are incubated with Jeko (with or without pretreatment with tafasitamab), at 1:5 ratio, and in the presence of CD28, CD49d and monensin (per standard degranulation methodology), CART19 continue to show similar levels of degranulation and cytokine production despite CD19 binding with the monoclonal antibody (n=2 independent experiments). GM-CSF, INFγ, IL-2 and MIP1b cytokine production levels did not differ between absence and presence of tafasitamab (data not shown).

FIG. 5: CART19 cells exhibit significant antigen specific proliferation unimpaired by the presence of tafasitamab. When CART19 or UTD were co-cultured with the CD19+ Jeko cells, at 1:1 ratios, in the presence or absence of tafasitamab, CART19 but not UTD exhibited significant antigen specific proliferation that was not different when CD19 was blocked by tafasitamab.

FIG. 6: DLBCL Case study: anti-CD19 CAR-T cell therapy is feasible after tafasitamab+lenalidomide

FIG. 7: Tumors were induced by injection of JeKo-1 cells into NGS mice at day −14. At day −8, mice were randomized into tafasitamab group (10 mice) and PBS group (4 mice, Δ). The tafasitamab group received 10 mg/kg 3×/week of tafasitamab via i.p. injection. At day −1, mice that received tafasitamab were randomized into tafasitamab continuation (o) or tafasitamab discontinuation (□) group. At day 0, all of the mice from 3 groups received 2.5x10 6 of CART19 cells (IV). Tafasitamab continuation group showed reduced survival compared with tafasitamab discontinuation or PBS control (**p=0.005, log-rank test, continuation vs discontinuation or continuation vs PBS, no significant difference between PBS group and tafasitamab discontinuation group).

FIG. 8: Tumors were induced by injection of JeKo-1 cells into NGS mice at day −14. At day −8, mice were randomized into tafasitamab group (10 mice) and PBS group (4 mice, Δ). The tafasitamab group received 10 mg/kg 3×/week of tafasitamab via ip injection. At day −1, mice that received tafasitamab were randomized into tafasitamab continuation (O) or discontinuation (□) group. At day 0, all of the mice from 3 groups received 2.5×10⁶ of CART19 cells (IV). (A) Tafasitamab continuation group showed higher tumor burden compared with discontinuation or PBS control (****p<0.0001, two-way ANOVA, continuation vs discontinuation or continuation vs PBS). (B) No difference was observed between tafasitamab discontinuation group and PBS group (n.s. not significant, two-way ANOVA, discontinuation vs. PBS).

### DEFINITIONS

The term “antibody” means monoclonal antibodies, including any isotype, such as, IgG, IgM, IgA, IgD and IgE. An IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are referred to as CDR1, CDR2, and CDR3, numbered sequentially from the N-terminus. The more highly conserved portions of the variable regions outside of the CDRs are called the “framework regions”. An “antibody fragment” means an Fv, scFv, dsFv, Fab, Fab′ F(ab′)2 fragment, or other fragment, which contains at least one variable heavy or variable light chain, each containing CDRs and framework regions.

“VH” refers to the variable region of an immunoglobulin heavy chain of an antibody, or antibody fragment. “VL” refers to the variable region of the immunoglobulin light chain of an antibody, or antibody fragment.

The term “CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12. Human CD19 (UniProt—P15391) has the amino acid sequence of

MPPPRLLFFLLFLTPMEVRPEEPLWKVEEGDNAVLQCLKGTSDGPTQQLTWSRESPLKPFLK LSLGLPGLGIHMRPLAIWLFIFNVSQQMGGFYLCQPGPPSEKAWQPGVVTVNVEGSGELFRWN VSDLGGLGCGLKNRSSEGPSSPSGKLMSPKLYVWAKDRPEIWEGEPPCLPPRDSLNQSLSQD LTMAPGSTLWLSCGVPPDSVSRGPLSWTHVHPKGPKSLLSLELKDDRPARDMWVMETGLLLP RATAQDAGKYYCHRGNLTMSFHLEITARPVLWHWLLRTGGWKVSAVTLAYLIFCLCSLVGILHL QRALVLRRKRKRMTDPTRRFFKVTPPPGSGPQNQYG NVLSLPTPTSGLGRAQRWAAGLGGT APSYGNPSSDVQADGALGSRSPPGVGPEEEEGEGYEEPDSEEDSEFYENDSNLGQDQLSQD GSGYENPEDEPLGPEDEDSFSNAESYENEDEELTQPVARTMDFLSPHGSAWDPSREATSLGS QSYEDMRGILYAAPQLRSIRGQPGPNHEEDADSYENMDNPDGPDPAWGGGGRMGTWSTR (SEQ ID NO:13). Variants of human CD19 (e.g. splice variants, polymorphisms and SNP) are also encompassed by this application.

“MOR00208”, “MOR208”, “XmAb 5574” or “tafasitamab” is an anti-CD19 antibody. The amino acid sequence of the VH and VL domain are given in SEQ ID NO: 7 and SEQ ID NO: 8, respectively. The amino acids sequence of the MOR208 heavy chain Fc region is:

(SEQ ID NO: 9)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPDVFLFPPKPKDTLMISRTPEVTCVVVDVS
HEDPEVQFNWYVDGVEVHNAKTKPREEQFNSTFRVVSVLTVVHQDWLNGK
EYKCKVSNKALPAPEEKTISKTKGQPREPQVYTLPPSREEMTKNQVSLTC
LVKGFYPSDIAVEWESNGQPENNYKTTPPMLDSDGSFFLYSKLTVDKSRW
QQGNVFSCSVMHEALHNHYTQKSLSLSPGK.

The amino acids sequence of the MOR208 light chain Fc region is: RTVAAPSVFIFPPSDEQLKSGTASVVCLLNNFYPREAKVQWKVDNALQSGNSQESVTEQDSK DSTYSLSSTLTLSKADYEKHKVYACEVTHQGLSSPVTKSFNRGEC (SEQ ID NO: 10). The MOR208 antibody is described in U.S. patent application Ser. No. 12/377,251, which is incorporated by reference in its entirety as antibody named 4G7 H1.52 Hybrid S239D/I332E/4G7 L1.155 (later named MOR00208) as follows:

>4G7 H1.52 Hybrid S239D/1332E
(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K
> 4G7 L1.155
(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestable solution, capsule or tablet. Preferably, the administration is by an injectable form.

The term “chimeric antigen receptors (CARs),” as used herein, may refer to engineered T-cell receptors, chimeric T-cell receptors, or chimeric immunoreceptors, for example, and encompass engineered receptors that graft a defined specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor-associated antigen, for example. In some embodiments, CARs comprise an intracellular activation domain, a transmembrane domain, and an extracellular domain comprising a tumor-associated antigen-binding region. In some aspects, CARs comprise fusions of FMC63 single-chain variable fragments (scFv) derived from monoclonal antibodies fused to a hinge linker CD8h, transmembrane domain CD8TM, and a signaling endodomain 41BBζ. The specificity of CAR designs may be be derived from antibody fragments (e.g. scFv, Fab, VHH, scFab), ligands of receptors (e.g., peptides) or from Dectins. In specific embodiments, one can target malignant B cells by redirecting the specificity of T cells by using a CAR specific for the B-lineage molecule, CD19. In certain cases, CARs comprise domains for additional co-stimulatory signaling, such as but not limited to CD3ζ, FcR, CD27, CD28, CD137, DAP10, 41BBζ and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, cytokines, and cytokine receptors.

A “therapeutically effective amount” of a compound or combination refers to an amount sufficient to cure, alleviate or partially arrest the clinical manifestations of a given disease or disorder and its complications. The amount that is effective for a particular therapeutic purpose will depend on the severity of the disease or injury as well as on the weight and general state of the subject. It is understood that determination of an appropriate dosage may be achieved, using routine experimentation, by constructing a matrix of values and testing different points in the matrix, all of which is within the ordinary skills of a trained physician or clinical scientist.

The term “hematologic cancer” includes blood-borne tumors and diseases or disorders involving abnormal cell growth and/or proliferation in tissues of hematopoietic origin, such as lymphomas, leukemias, and myelomas.

Non-Hodgkin's lymphoma (“NHL”) is a heterogeneous malignancy originating from lymphocytes. In the United States, the incidence is estimated at 65,000/year with mortality of approximately 20,000 (American Cancer Society, 2006; and SEER Cancer Statistics Review). The disease can occur in all ages, the usual onset begins in adults over 40 years, with the incidence increasing with age. NHL is characterized by a clonal proliferation of lymphocytes that accumulate in the lymph nodes, blood, bone marrow and spleen, although any major organ may be involved. The current classification system used by pathologists and clinicians is the World Health Organization (WHO) Classification of Tumours, which organizes NHL into precursor and mature B-cell or T-cell neoplasms. The PDQ is currently dividing NHL as indolent or aggressive for entry into clinical trials. The indolent NHL group is comprised primarily of follicular subtypes, small lymphocytic lymphoma, MALT (mucosa-associated lymphoid tissue), and marginal zone; indolent encompasses approximately 50% of newly diagnosed B-cell NHL patients. Aggressive NHL includes patients with histologic diagnoses of primarily diffuse large B cell lymphoma (DLBL, “DLBCL”, or DLCL) (40% of all newly diagnosed patients have diffuse large cell lymphoma), Burkitt's, and mantle cell lymphoma (“MCL”). The most commonly used agents for combination chemotherapy include cyclophosphamide, vincristine and prednisone (CVP); or cyclophosphamide, adriamycin, vincristine, prednisone (CHOP). Approximately 70% to 80% of patients will respond to their initial chemotherapy, duration of remissions last about 2-3 years. Ultimately, the majority of patients relapse. The discovery and clinical use of the anti-CD20 antibody, rituximab, has provided significant improvements in response and survival rate. The current standard of care for most patients is rituximab+CHOP (R-CHOP) or rituximab+CVP (R-CVP). Rituximab therapy has been shown to be efficacious in several types of NHL, and is currently approved as a first line treatment for both indolent (follicular lymphoma) and aggressive NHL (diffuse large B cell lymphoma). However, there are significant limitations of anti-CD20 monoclonal antibody (mAb), including primary resistance (50% response in relapsed indolent patients), acquired resistance (50% response rate upon re-treatment), rare complete response (2% complete response rate in relapsed population), and a continued pattern of relapse. Finally, many B cells do not express CD20, and thus many B-cell disorders are not treatable using anti-CD20 antibody therapy.

Chronic lymphocytic leukemia (also known as “chronic lymphoid leukemia” or “CLL”), is a type of adult leukemia caused by an abnormal accumulation of B lymphocytes. In CLL, the malignant lymphocytes may look normal and mature, but they are not able to cope effectively with infection. CLL is the most common form of leukemia in adults. Men are twice as likely to develop CLL as women. However, the key risk factor is age. Over 75% of new cases are diagnosed in patients over age 50. More than 10,000 cases are diagnosed every year and the mortality is almost 5,000 a year (American Cancer Society, 2006; and SEER Cancer Statistics Review). CLL is an incurable disease but progresses slowly in most cases. Many people with CLL lead normal and active lives for many years. Because of its slow onset, early-stage CLL is generally not treated since it is believed that early CLL intervention does not improve survival time or quality of life. Instead, the condition is monitored over time. Initial CLL treatments vary depending on the exact diagnosis and the progression of the disease. There are dozens of agents used for CLL therapy. Combination chemotherapy regimens such as FCR (fludarabine, cyclophosphamide and rituximab), and BR (Ibrutinib and rituximab) are effective in both newly-diagnosed and relapsed CLL. Allogeneic bone marrow (stem cell) transplantation is rarely used as a first-line treatment for CLL due to its risk.

Another type of leukemia is Small lymphocytic lymphoma (“SLL”) that is considered a CLL variant that lacks the clonal lymphocytosis required for the CLL diagnosis, but otherwise shares pathological and immunophenotypic features (Campo et al., 2011). The definition of SLL requires the presence of lymphadenopathy and/or splenomegaly. Moreover, the number of B lymphocytes in the peripheral blood should not exceed 5E+09/L. In SLL, the diagnosis should be confirmed by histopathologic evaluation of a lymph node biopsy whenever possible (Hallek et al., 2008). The incidence of SLL is approximately 25% of CLL in the US (Dores et al., 2007).

Another type of leukemia is acute lymphoblastic leukemia (ALL), also known as acute lymphocytic leukemia. ALL is characterized by the overproduction and continuous multiplication of malignant and immature white blood cells (also known as lymphoblasts) in the bone marrow. ‘Acute’ refers to the undifferentiated, immature state of the circulating lymphocytes (“blasts”), and that the disease progresses rapidly with life expectancy of weeks to months if left untreated.

“Subject” or “patient” as used in this context refers to any mammal, including rodents, such as mouse or rat, and primates, such as cynomolgus monkey (Macaca fascicularis), rhesus monkey (Macaca mulatta) or humans (Homo sapiens). Preferably, the subject or patient is a primate, most preferably a human.

The terms “combination” or “pharmaceutical combination” refer to the administration of one therapy in addition to another therapy. As such, “in combination with” includes simultaneous (e.g., concurrent) and consecutive administration in any order. By way of non-limiting example, a first therapy (e.g., agent, such as an anti-CD19 antibody) may be administered before (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., pharmaceutical agent, such as lenalidomide) to a patient.

The term “sequential combination” or “sequential therapeutic combination” refers to the administration of a different therapy after a previous therapy was completed. Additional therapies may be included between the two therapies. For example and as an embodiment as disclsoed herein a sequential combination refers to the treatment of a patient with a chimeric antigen receptor (CAR) T cell directed against CD19 after the patient has relapsed from or is refractory to a previous therapy comprising an anti-CD19 antibody as disclosed herein. In an embodiment the previous or first therapy of a sequential combination was completed for 1 week, 2 weeks, 3 weeks, 4 weeks, 5 weeks, 6 weeks, 7 weeks, 8 weeks, 9 weeks, 10 weeks, 11 weeks, or 12 weeks or longer before the second different therapy is administered.

A “thalidomide analog” includes, but is not limited to, thalidomide itself, lenalidomide (CC-5013, Revlimid™), Pomalidomide (CC4047, Actimid™) and the compounds disclosed in WO2002068414 and WO2005016326, which are incorporated by reference in their entireties. The term refers to a synthetic chemical compound using the thalidomide structure as a backbone (e.g., side groups have been added or such groups have been deleted from the parent structure). The analog differs in structure from thalidomide and its metabolite compounds such as by a difference in the length of an alkyl chain, a molecular fragment, by one or more functional groups, or change in ionization. The term “thalidomide analog” also includes the metabolites of thalidomide. Thalidomide analogs include the racemic mixture of the S- and the R-enantiomer of a respective compound and the S-enantiomer or to the R-enantiomer individually. The racemic mixture is preferred. Thalidomide analogs include compounds such as lenalidomide which has the following structure:

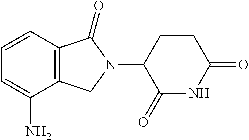 [SMILES: Nc1cccc2c1CN(C1CCC(=O)NC1=O)C2=O]

The term “relapse” as used herein refers to reappearance of a disease (e.g. cancer) after an initial period of responsiveness, e.g. after prior treatment with a therapy, e.g., cancer therapy (e.g. complete response or partial response). More generally, in an embodiment, a response (e.g. complete response or partial response) can involve the absence of detectable MRD (minimal residual disease). In an embodiment, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

“Refractory” as used herein refers to a disease, e.g., cancer that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

TABLE 1
Sequence Listing

| | SEQ ID NO: | Amino Acids |
|---|---|---|
| HCDR1 | SEQ ID NO: 1 | SYVMH |
| HCDR2 | SEQ ID NO: 2 | NPYNDG |
| HCDR3 | SEQ ID NO: 3 | GTYYYGTRVFDY |
| LCDR1 | SEQ ID NO: 4 | RSSKSLQNVNGNTYLY |
| LCDR2 | SEQ ID NO: 5 | RMSNLNS |
| LCDR3 | SEQ ID NO: 6 | MQHLEYPIT |
| VH | SEQ ID NO: 7 | EVQLVESGGGLVKPGGSLKLSCAASGYTFTSY |
| | | VMHWVRQAPGKGLEWIGYINPYNDGTKYNEK |
| | | FQGRVTISSDKSISTAYMELSSLRSEDTAMYYC |
| | | ARGTYYYGTRVFDYWG QGTLVTVSS |
| VL | SEQ ID NO: 8 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIK |
| Heavy chain constant | SEQ ID NO: 9 | ASTKGPSVFPLAPSSKSTSGGTAALGCLVKDY |
| domain | | FPEPVTVSWNSGALTSGVHTFPAVLQSSGLYS |
| | | LSSVVTVPSSSLGTQTYICNVNHKPSNTKVDK |
| | | KVEPKSCDKTHTCPPCPAPELLGGPDVFLFPP |
| | | KPKDTLMISRTPEVTCVVVDVSHEDPEVQFNW |
| | | YVDGVEVHNAKTKPREEQFNSTFRVVSVLTVV |
| | | HQDWLNGKEYKCKVSNKALPAPEEKTISKTKG |
| | | QPREPQVYTLPPSREEMTKNQVSLTCLVKGFY |
| | | PSDIAVEWESNGQPENNYKTTPPMLDSDGSF |
| | | FLYSKLTVDKSRWQQGNVFSCSVMHEALHNH |
| | | YTQKSLSLSPGK |
| Light chain constant | SEQ ID NO: 10 | RTVAAPSVFIFPPSDEQLKSGTASVVCLLNNFY |
| domain | | PREAKVQWKVDNALQSGNSQESVTEQDSKD |
| | | STYSLSSTLTLSKADYEKHKVYACEVTHQGLS |
| | | SPVTKSFNRGEC |
| Full Heavy chain | SEQ ID NO: 11 | EVQLVESGGGLVKPGGSLKLSCAASGYTFTSY |
| | | VMHWVRQAPGKGLEWIGYINPYNDGTKYNEK |
| | | FQGRVTISSDKSISTAYMELSSLRSEDTAMYYC |
| | | ARGTYYYGTRVFDYWGQGTLVTVSSASTKGP |
| | | SVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKS |
| | | CDKTHTCPPCPAPELLGGPDVFLFPPKPKDTL |
| | | MISRTPEVTCVVVDVSHEDPEVQFNWYVDGV |
| | | EVHNAKTKPREEQFNSTFRVVSVLTVVHQDW |
| | | LNGKEYKCKVSNKALPAPEEKTISKTKGQPRE |
| | | PQVYTLPPSREEMTKNQVSLTCLVKGFYPSDI |
| | | AVEWESNGQPENNYKTTPPMLDSDGSFFLYS |
| | | KLTVDKSRWQQGNVFSCSVMHEALHNHYTQK |
| | | SLSLSPGK |
| Full Light chain | SEQ ID NO: 12 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIKRTVAAPSVFIFPPSDEQ |
| | | LKSGTASVVCLLNNFYPREAKVQWKVDNALQ |
| | | SGNSQESVTEQDSKDSTYSLSSTLTLSKADYE |
| | | KHKVYACEVTHQGLSSPVTKSFNRGEC |
| Human CD19 | SEQ ID NO: 13 | MPPPRLLFFLLFLTPMEVRPEEPLVVKVEEGD |
| | | NAVLQCLKGTSDGPTQQLTWSRESPLKPFLKL |
| | | SLGLPGLGIHMRPLAIWLFIFNVSQQMGGFYLC |
| | | QPGPPSEKAWQPGWTVNVEGSGELFRWNVS |
| | | DLGGLGCGLKNRSSEGPSSPSGKLMSPKLYV |
| | | WAKDRPEIWEGEPPCLPPRDSLNQSLSQDLT |
| | | MAPGSTLWLSCGVPPDSVSRGPLSWTHVHPK |
| | | GPKSLLSLELKDDRPARDMWVMETGLLLPRAT |
| | | AQDAGKYYCHRGNLTMSFHLEITARPVLWHW |
| | | LLRTGGWKVSAVTLAYLIFCLCSLVGILHLQRA |
| | | LVLRRKRKRMTDPTRRFFKVTPPPGSGPQNQ |
| | | YGNVLSLPTPTSGLGRAQRWAAGLGGTAPSY |
| | | GNPSSDVQADGALGSRSPPGVGPEEEEGEG |
| | | YEEPDSEEDSEFYENDSNLGQDQLSQDGSGY |
| | | ENPEDEPLGPEDEDSFSNAESYENEDEELTQP |
| | | VARTMDFLSPHGSAWDPSREATSLGSQSYED |
| | | MRGILYAAPQLRSIRGQPGPNHEEDADSYEN |
| | | MDNPDGPDPAWGGGGRMGTWSTR |

In various aspects, the invention is directed to nucleic acid sequences encoding any of the antibodies or CARs disclosed for use in the treatment of cancer in a patient as described herein.

### WORKING EXAMPLES

CART19 cells used for experiments were generated through lentiviral transduction of healthy donor T cells with a second-generation CD19 CAR construct (Clone FMC63 as CD19 binding domain, FMC63-CD8h-CD8TM-41BEK, similar to the FDA approved construct used for tisagenlecleucel). T cells isolated from normal donors were stimulated using Cell Therapy Systems Dynabeads CD3/CD28 (Life Technologies, Oslo, Norway) at a 1:3 ratio (cells:beads) and then transduced 24 hours after stimulation with lentivirus particles at a MOI of 3.0. Magnetic bead removal and the evaluation of CAR19 expression on T cells by flow cytometry were performed on day 6. CART cells were harvested and cryopreserved on day 8 for future experiments. CART cells were thawed and rested in T cell medium 6-12 hours prior to their use in experiments, as specified in each experiment.

### Example 1

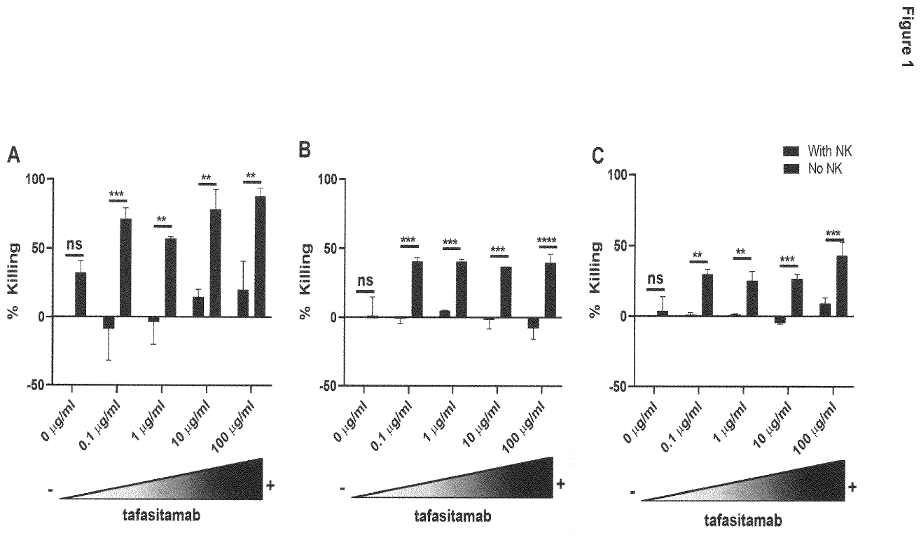

Functional activity of tafasitamab or CART19 cells, was tested on CD19 positive target cell lines, Jeko (mantel cell lymphoma), Ly7 (DLBCL) and Nalm-6 (ALL). Cell lines were originally obtained from ATCC or DSMZ. Cell lines were transduced with luciferase (firefly/EGFP, CBG/EGFP, or CBR/EGFP) and then sorted to obtain a greater than 99% positive population. In 24 h ADCC assays (tafasitamab titration plus natural killer (NK) cells; FIG. 1) and T cell cytotoxicity assays (CART19, E:T titrations; data not shown) distinct activity on all cell lines tested was observed for both therapies. Killing assays were performed according to standard protocols (CaoL-F et al. Cytometry A, 2010, vol. 77 6 (pg. 534-545). In brief, cytotoxicity was assessed 24 hours after incubation of the CD19+ luciferase+target cells with different concentrations of tafasitamab, in the presence or absence of NK cells.

### Example 2

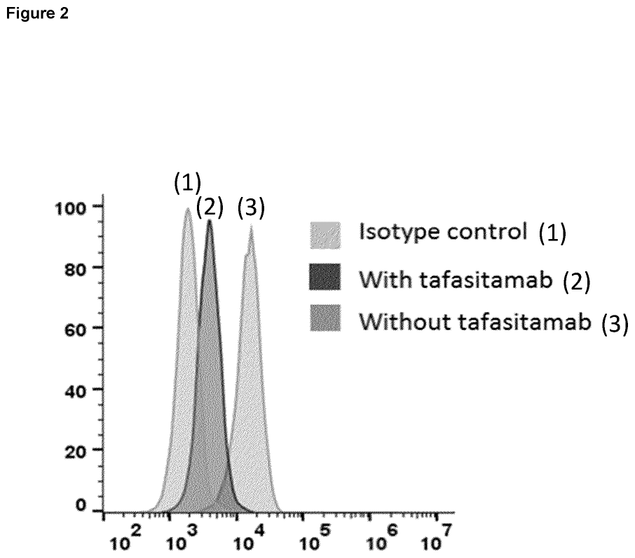

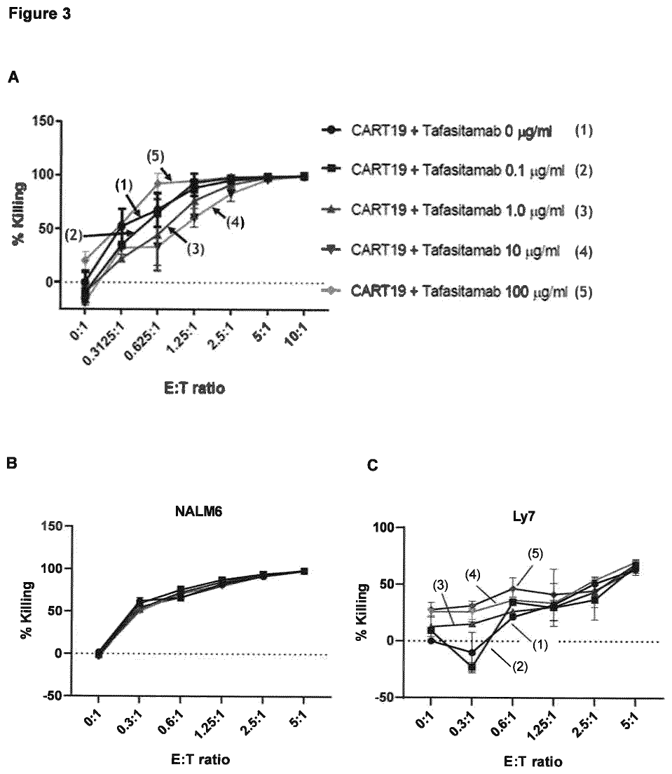

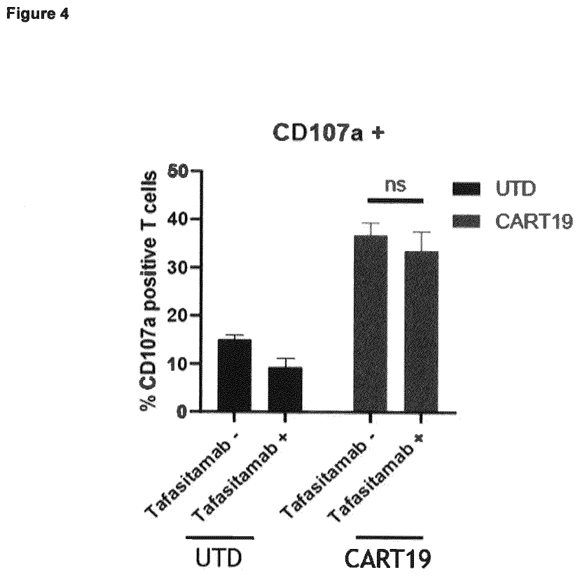

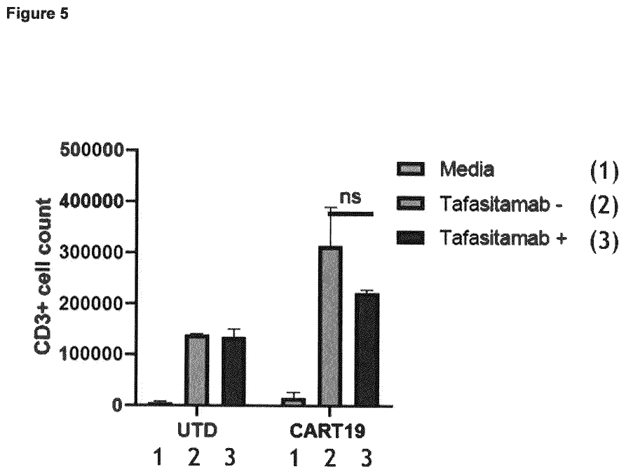

It was studied, whether the observed CART19 activity may be influenced by tafasitamab in case of a direct CD19 binding competition between tafasitamab and the CAR. To first test for such binding competition, the CD19+ cell lines Nalm-6 or Jeko were incubated with 2 or 0.5 mg/mL tafasitamab, to saturate the receptors. Subsequent flow cytometry analysis using the FMC63 antibody (carrying the same CD19 binding domain as CART19) failed to detect CD19 expression, indicating a direct binding competition between FMC63 and tafasitamab (FIG. 2). To investigate for any potential impact of such binding competition on CART19 cell effector functions, tafasitamab was incubated with target cells (CD19+ cell lines Jeko, Ly7 or Nalm-6) at increasing concentrations and then CART19 cells were added at different effector: target ratios (0.1:1 to 10:1), without other effector cells to the cell culture. The presence of tafasitamab, i.e. binding to the CD19 antigen, did not affect important CART cell effector functions such as antigen specific killing (FIG. 3), degranulation (FIG. 4), cytokine production or antigen specific proliferation of CART19 cells (FIG. 5).

In summary, the data indicate that CART19 cells continue to exhibit potent antigen specific effector functions despite presence of tafasitamab and its competition for CD19 binding. Thus, targeting of CD19 by tafasitamab does not impair CD19 directed chimeric antigen receptor (CAR) T cell activity in vitro.

### Example 3

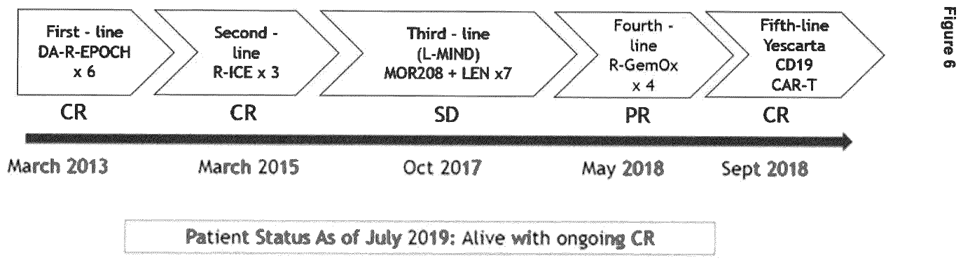

In a case study a 58-year-old female patient who initially presented with an 8 cm mesenteric mass, workup revealed stage III germinal center B cell—like (GCB) DLBCL arising from follicular lymphoma, Ki-67 proliferation index 80%, and IGH/BCL2 fusion. The patient received 6 cycles of dose-adjusted R-EPOCH. Despite achieving a complete remission (CR) to this frontline therapy, the patient experienced disease relapse within two years. Subsequently, the patient received rituximab plus ifosfamide, carboplatin, and etoposide (RICE) chemotherapy, to which a second CR was achieved. The patient declined ASCT. Second relapse occurred approximately two years later. After meeting eligibility criteria, she was enrolled in the L-MIND trial and received TAFA plus LEN for 6 cycles (1 cycle=28 days; TAFA 12 mg/kg intravenously, weekly×3 cycles, biweekly thereafter; LEN 25 mg daily on days 1-21 of each cycle). TAFA/LEN was well tolerated. Stable disease was achieved with this investigational regimen, followed by progression 6 month later. Fourth-line treatment consisted of rituximab, gemcitabine, oxaliplatin, (R-GEM-OX) for 4 cycles, to which the patient had a partial response. Shortly thereafter, the patient received CAR T therapy directed against CD19 (axicabtagene ciloleucel [YESCARTA®]) (FIG. 6). Treatment course was complicated by grade 2 cytokine release syndrome. A complete response was achieved one month after treatment, and has been sustained since then. As of the present date the patient remains without clinical evidence of relapse. Thus, a sustained remission is achieved from CAR-T cell therapy directed against CD19, despite prior treatment with tafasitamab in a patient with relapsed and refractory Diffuse Large B-Cell Lymphoma (R/R-DLBCL).

The half-life of tafasitamab is approximately 16 days, suggesting that it was eliminated prior to CAR T-cell infusion five months later. Although a biopsy was not done upon progression after TAFA, it is assumed that CD19 antigen escape did not account for the relapse, since the patient has achieved sustained remission with subsequent CAR-T cell therapy directed against CD19. Therefore, disease progression following treatment with anti-CD19 monoclonal antibody tafasitamab may not preclude patients from CAR-T cell therapy directed against CD19, despite previously targeting the same antigen.

### Example 4

NSG (NOD scid gamma mouse, immunodeficient laboratory mice) mice were injected with 1×10⁶ luciferase+JeKo-1 cells at day −14. At day −8, tumor burden was assessed with bioluminescence imaging (BLI) and mice were randomized into tafasitamab group (10 mice) and PBS group (4 mice). The tafasitamab group received 10 mg/kg 3×/week of tafasitamab via ip injection. At day −1, tumor burden was assessed by BLI and mice were randomized into tafasitamab continuation or discontinuation group. At day 0, all of the mice from 3 groups received 2.5×10⁶ of CART19 cells (IV). Those mice from the tafasitamab continuation group continued the tafasitamb treatment in parallel to the CART19 cells. For all mice, BLI to monitor tumor load was performed weekly and survival of mice was monitored. Mice were sacrificed

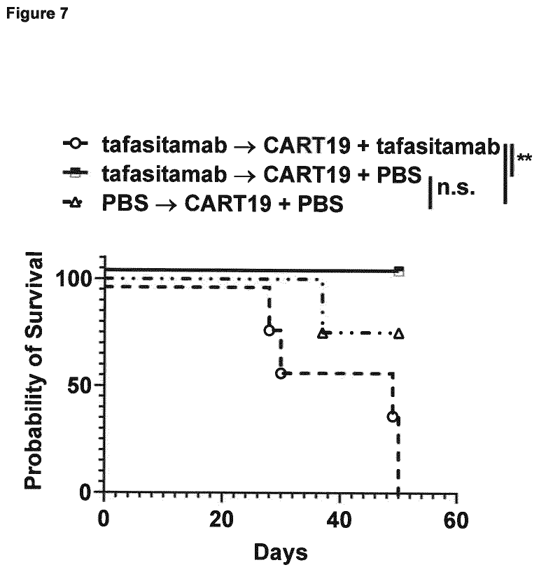

CD19 competition was observed in the tafasitamab continuation group and demonstrated shorter survival periods (p=0.005, log-rank test, continuation vs discontinuation or continuation vs PBS). However, the tafasitamab discontinuation group led mice into remission and showed no reduced survival. Overall and regarding overall survival, there was no difference between PBS group and tafasitamab discontinuation group (log-rank test) (FIG. 7).

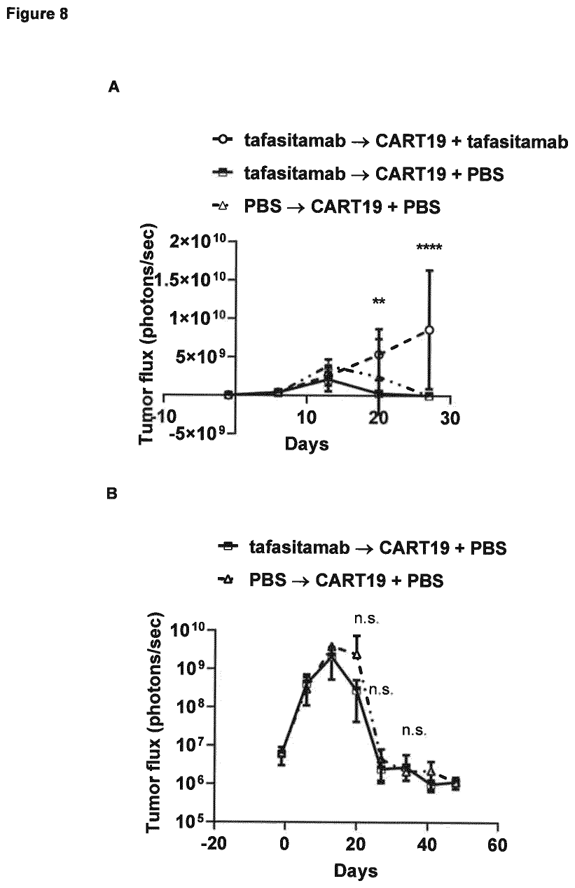

Furthermore, the tafasitamab continuation group showed higher tumor burden compared with discontinuation group or PBS control group (****p<0.0001, two-way ANOVA, continuation vs discontinuation or continuation vs PBS). No difference was observed between tafasitamab discontinuation group and PBS group (n.s. not significant, two-way ANOVA, discontinuation vs. PBS) (FIG. 8).

Therefore, these in vivo data indicate that CAR-T cell therapy directed against CD19 is not impaired by previous tafasitamab treatment. It can be concluded that previous tafasitamab therapy does not preclude patients from CAR-T cell therapy directed against CD19.

However, these data also indicate that in case the CD19 antibody and the CD19 CARTs compete for the same or overlapping CD19 epitope a combined parallel treatment of such CD19 antibody and CAR-T cell therapy directed against CD19 might negatively affect efficacy of such therapies.

### EMBODIMENTS

In one embodiment, a therapeutic agent directed against CD19 is disclosed for use in the treatment of cancer in a patient, wherein the patient was previously treated with a composition comprising an anti-CD19 antibody, the antibody comprising an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6).

In one aspect, a therapeutic agent directed against CD19 is disclosed for use in the treatment of cancer in a patient, wherein the patient is refractory or relapsed from previous treatment, the previous treatment comprising an anti-CD19 antibody comprising an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6).

In another embodiment, a therapeutic agent directed against CD19 is disclosed for use in the treatment of cancer in a patient, wherein the patient is refractory or relapsed from previous treatment, the previous treatment comprising an anti-CD19 antibody comprising a variable heavy chain comprising the sequence

EVQLVESGGGLVKPGGSLKLSCAASGYTFTSYVMHVVVRQAPGKGLEWIGYINPYNDGTKYNE KFQGRVTISSDKSISTAYMELSSLRSEDTAMYYCARGTYYYGTRVFDYWGQGTLVTVSS (SEQ ID NO: 7) and/or comprising a variable light chain of the sequence

DIVMTQSPATLSLSPGERATLSCRSSKSLQNVNGNTYLYWFQQKPGQSPQLLIYRMSNLNSGV PDRFSGSGSGTEFTLTISSLEPEDFAVYYCMQHLEYPITFGAGTKLEIK (SEQ ID NO: 8)

In one embodiment, a therapeutic agent directed against CD19 is disclosed for use in the treatment of cancer in a patient, wherein the patient is refractory or relapsed from previous treatment, the previous treatment comprising an anti-CD19 antibody, the antibody comprising a variable heavy chain and/or a variable light chain with at least 90%, at least 91%, at least 92%, at least 93%, at least 94%, at least 95%, at least 96%, at least 97%, at least 98% or at least 99% sequence identity to the variable heavy chain of SEQ ID NO: 7 and/or the variable light chain of SEQ ID NO: 8.

In a preferred embodiment, a therapeutic agent directed against CD19 is disclosed for use in the treatment of cancer in a patient, wherein the patient is refractory or relapsed from previous treatment, the previous treatment comprising an anti-CD19 antibody, the antibody comprising a heavy chain of SEQ ID NO: 11 and a light chain of SEQ ID NO: 12.

In another preferred embodiment, a therapeutic agent directed against CD19 is disclosed for use in the treatment of cancer in a patient, wherein the patient is refractory or relapsed from previous treatment, the previous treatment comprising the anti-CD19 antibody tafasitamab.

In another embodiment, a therapeutic agent directed against CD19 is disclosed for use in the treatment of cancer in a patient, wherein the patient is refractory or relapsed from previous treatment, the previous treatment comprising an anti-CD19 antibody that is competing with the therapeutic agent directed against CD19 for binding to human CD19.

In other embodiments, a therapeutic agent directed against CD19 is disclosed for use in the treatment of cancer in a patient, wherein the patient is refractory or relapsed from previous treatment, the previous treatment comprising an anti-CD19 antibody, and wherein the therapeutic agent directed against CD19 is selected from the group of an antibody, an antibody-drug conjugate, a bispecific, an alternative scaffold protein or a chimeric antigen receptor (CAR) T cell.

In a preferred embodiment, the therapeutic agent directed against CD19 for use in the treatment of cancer in a patient, wherein the patient is refractory or relapsed from previous treatment comprising an anti-CD19 antibody, is a chimeric antigen receptor (CAR) T cell. In certain embodiments, the CAR-T cell is preferably one of or a combination of two or more of axicabtagen-ciloleucel (KTE-C19, Axi-cel, YESCARTA®) and/or tisagenlecleucel (CTL019, KYMRIAH®), lisocabtagene maraleucel (JCAR017, clinical trials: e.g. NCT02631044, NCT03310619), or UCARTI9 (S 68587, NCT02808442). In another aspect, the CAR-T cell is one of or a combination of two or more of CAR-T cell constructs known as KITE037, welgenaleucel, ICTCAR-003, IM-19, CTX-110, SSCAR-010, ICTCAR-011.

In other embodiments, the therapeutic agent is directed against a B cell lineage marker for use in the treatment of cancer in a patient, wherein the patient is refractory or relapsed from previous treatment, the previous treatment comprising an anti-CD19 antibody. Preferably, the therapeutic agent is directed against CD19 and/or CD20 for use in the treatment of cancer in a patient, wherein the patient is refractory or relapsed from previous treatment, the previous treatment comprising the anti-CD19 antibody tafasitamab.

In another embodiment, the CAR-T cell is directed against CD19 and/or CD20 for use in the treatment of cancer in a patient, wherein the patient is refractory or relapsed from previous treatment, the previous treatment comprising the anti-CD19 antibody tafasitamab.

In preferred embodiments, said therapeutic agent is for use in a pharmaceutical composition. In other embodiments, the therapeutic agent is comprised in a pharmaceutical composition.

In one aspect, a therapeutic agent directed against CD19 is disclosed for use in the treatment of cancer in a patient, wherein the patient is refractory or relapsed from previous treatment, the previous treatment comprising an anti-CD19 antibody and wherein the cancer is a hematological cancer. In certain embodiments, the hematological cancer is selected from non-Hodgkin's B cell lymphoma, such as for example follicular lymphoma (FL), small lymphocytic lymphoma (SLL), Burkitt lymphoma and diffuse large B-cell lymphoma (DLBCL), Waldenstrom's macroglobulinemia, leukemia, such as for example chronic lymphocytic leukemia (CLL), acute lymphoblastic leukemia (ALL) or acute myeloid leukemia (AML). In a preferred embodiment, the hematological cancer is DLBCL. In other embodiments, the cancer is relapsed or refractory (R/R) cancer, preferably a relapsed or refractory hematological cancer. The R/R hematological cancer is selected from R/R non-Hodgkin's B cell lymphoma, such as for example R/R follicular lymphoma (FL), R/R small lymphocytic lymphoma (SLL), R/R Burkitt lymphoma and R/R diffuse large B-cell lymphoma (DLBCL), R/R Waldenstrom's macroglobulinemia, R/R leukemia, such as for example R/R chronic lymphocytic leukemia (CLL), R/R acute lymphoblastic leukemia (ALL) or R/R acute myeloid leukemia (AML). In a preferred embodiment, the R/R hematological cancer is relapsed or refractory (R/R) Diffuse Large B-Cell Lymphoma (DLBCL).

In a particular embodiment, a CAR-T cell directed against CD19 is disclosed for use in the treatment of R/R DLBCL in a patient, wherein the patient is refractory or relapsed from previous treatment, the previous treatment comprising an anti-CD19 antibody, preferably an antibody comprising
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab.

In a particular embodiment, a CAR-T cell directed against CD19 is disclosed for use in the treatment of R/R DLBCL in a patient, wherein the patient is refractory or relapsed from previous treatment, the previous treatment comprising a combination of tafasitamab and Ienalidomide.

In another embodiment, a CAR-T cell directed against CD19 is disclosed for use in the treatment of R/R DLBCL in a patient, wherein the patient is refractory or relapsed from previous treatment, the previous treatment comprising a combination of tafasitamab and bendamustine.

In one aspect, an anti-CD19 antibody is disclosed herein, for use in the treatment of cancer in a patient, wherein said patient after such anti-CD19 antibody treatment is treated with a CAR-T cell directed against CD19 and wherein the anti-CD19 antibody comprises an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6).

In one aspect, an anti-CD19 antibody is disclosed herein, for use in the treatment of cancer in a patient, wherein said patient after such anti-CD19 antibody treatment is treated with a CAR-T cell directed against CD19 and wherein the anti-CD19 antibody comprises a variable heavy chain of SEQ ID NO: 7 and a variable light chain of SEQ ID NO: 8.

In one aspect, an anti-CD19 antibody is disclosed herein, for use in the treatment of cancer in a patient, wherein said patient after such anti-CD19 antibody treatment is treated with a CAR-T cell directed against CD19 and wherein the anti-CD19 antibody comprises a heavy chain of SEQ ID NO: 11 and a light chain of SEQ ID NO: 12.

In a particular aspect, an anti-CD19 antibody is disclosed herein, for use in the treatment of cancer in a patient, wherein said patient after such anti-CD19 antibody treatment is treated with a CAR-T cell directed against CD19 and wherein the anti-CD19 antibody comprises tafasitamab.

In one aspect, an anti-CD19 antibody is disclosed herein, for use in the treatment of cancer in a patient, wherein said patient after such anti-CD19 antibody treatment is refractory or relapsed and is treated with a CAR-T cell directed against CD19 and wherein the anti-CD19 antibody comprises an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6).

In one aspect, an anti-CD19 antibody is disclosed herein, for use in the treatment of cancer in a patient, wherein said patient after such anti-CD19 antibody treatment is refractory or relapsed and is treated with a CAR-T cell directed against CD19 and wherein the anti-CD19 antibody comprises a variable heavy chain of SEQ ID NO: 7 and a variable light chain of SEQ ID NO: 8.

In one aspect, an anti-CD19 antibody is disclosed herein, for use in the treatment of cancer in a patient, wherein said patient after such anti-CD19 antibody treatment is refractory or relapsed and is treated with a CAR-T cell directed against CD19 and wherein the anti-CD19 antibody comprises a heavy chain of SEQ ID NO: 11 and a light chain of SEQ ID NO: 12.

In a particular aspect, an anti-CD19 antibody is disclosed herein, for use in the treatment of cancer in a patient, wherein said patient after such anti-CD19 antibody treatment is refractory or relapsed and is treated with a CAR-T cell directed against CD19 and wherein the anti-CD19 antibody comprises tafasitamab.

In one aspect, an anti-CD19 antibody is disclosed herein, for use in the treatment of cancer in a patient, wherein said patient after such anti-CD19 antibody treatment in combination with lenalidomide or bendamustine is refractory or relapsed, and is treated with a CAR-T cell directed against CD19 and wherein the anti-CD19 antibody comprises an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6).

In one aspect, an anti-CD19 antibody is disclosed herein, for use in the treatment of cancer in a patient, wherein said patient after such anti-CD19 antibody treatment in combination with lenalidomide or bendamustine is refractory or relapsed, and is treated with a CAR-T cell directed against CD19 and wherein the anti-CD19 antibody comprises a variable heavy chain of SEQ ID NO: 7 and a variable light chain of SEQ ID NO: 8.

In one aspect, an anti-CD19 antibody is disclosed herein, for use in the treatment of cancer in a patient, wherein said patient after such anti-CD19 antibody treatment in combination with lenalidomide or bendamustine is refractory or relapsed, and is treated with a CAR-T cell directed against CD19 and wherein the anti-CD19 antibody comprises a heavy chain of SEQ ID NO: 11 and a light chain of SEQ ID NO: 12.

In a particular aspect, an anti-CD19 antibody is disclosed herein, for use in the treatment of cancer in a patient, wherein said patient after such anti-CD19 antibody treatment in combination with lenalidomide or bendamustine is refractory or relapsed, and is treated with a CAR-T cell directed against CD19 and wherein the anti-CD19 antibody comprises tafasitamab.

In one aspect, an anti-CD19 antibody is disclosed herein, for use in the treatment of cancer in a patient, wherein said patient after such anti-CD19 antibody treatment is refractory or relapsed and is treated with a CAR-T cell directed against CD19 and wherein the anti-CD19 antibody comprises an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), and wherein the CAR-T cell directed against CD19 comprises one of or a combination of two or more of axicabtagen-ciloleucel (YESCARTA®) and/or tisagenlecleucel (KYMRIAH®), lisocabtagene maraleucel (JCAR017), or UCART19 (S 68587).

In one aspect, an anti-CD19 antibody is disclosed herein, for use in the treatment of cancer in a patient, wherein said patient after such anti-CD19 antibody treatment is refractory or relapsed and is treated with a CAR-T cell directed against CD19 and wherein the anti-CD19 antibody comprises a variable heavy chain of SEQ ID NO: 7 and a variable light chain of SEQ ID NO: 8, and wherein the CAR-T cell directed against CD19 comprises one of or a combination of two or more of axicabtagen-ciloleucel (YESCARTA®) and/or tisagenlecleucel (KYMRIAH®), lisocabtagene maraleucel (JCAR017), or UCART19 (S 68587).

In one aspect, an anti-CD19 antibody is disclosed herein, for use in the treatment of cancer in a patient, wherein said patient after such anti-CD19 antibody treatment is refractory or relapsed and is treated with a CAR-T cell directed against CD19 and wherein the anti-CD19 antibody comprises a heavy chain of SEQ ID NO: 11 and a light chain of SEQ ID NO: 12, and wherein the CAR-T cell directed against CD19 comprises one of or a combination of two or more of axicabtagen-ciloleucel (YESCARTA®) and/or tisagenlecleucel (KYMRIAH®), lisocabtagene maraleucel (JCAR017), or UCART19 (S 68587).

In a particular aspect, an anti-CD19 antibody is disclosed herein, for use in the treatment of cancer in a patient, wherein said patient after such anti-CD19 antibody treatment is refractory or relapsed and is treated with a CAR-T cell directed against CD19 and wherein the anti-CD19 antibody comprises tafasitamab, and wherein the CAR-T cell directed against CD19 comprises one of or a combination of two or more of axicabtagen-ciloleucel (YESCARTA®) and/or tisagenlecleucel (KYMRIAH®), lisocabtagene maraleucel (JCAR017), or UCART19 (S 68587).

In other aspects, the CAR-T cell is one of or a combination of two or more of CAR-T cell constructs known as KITE037, welgenaleucel, ICTCAR-003, IM-19, CTX-110, SSCAR-010, ICTCAR-011

In another aspect, a sequential therapeutic combination is disclosed comprising an anti-CD19 antibody and a chimeric antigen receptor (CAR) T cell directed against CD19 agent for use in the treatment of cancer.

In a certain aspect, a sequential therapeutic combination is disclosed comprising an anti-CD19 antibody and a CAR-T cell directed against CD19 for use in the treatment of cancer, wherein the anti-CD19 antibody is administered to a cancer patient and wherein after said patient has relapsed or is refractory, a CAR-T cell directed against CD19 is administered to the patient.

In another aspect, a sequential therapeutic combination is disclosed comprising an anti-CD19 antibody and a CAR-T cell directed against CD19 for use in the treatment of cancer, wherein the anti-CD19 antibody is administered to a cancer patient and wherein after said patient has relapsed or is refractory, a CAR-T cell directed against CD19 is administered to the patient and wherein the anti-CD19 antibody is administered at least bi-weekly.

In a further aspect, a sequential therapeutic combination is disclosed comprising an anti-CD19 antibody and a CAR-T cell directed against CD19 for use in the treatment of cancer, wherein the anti-CD19 antibody is administered to a cancer patient and wherein after said patient has relapsed or is refractory, a CAR-T cell directed against CD19 is administered to the patient, and wherein the anti-CD19 antibody is administered in combination with one or more additional pharmaceutical agent.

In another aspect, a sequential therapeutic combination is disclosed comprising an anti-CD19 antibody and a CAR-T cell directed against CD19 for use in the treatment of cancer, wherein the anti-CD19 antibody is administered at least biweekly to a cancer patient and wherein after said patient has relapsed or is refractory, a CAR-T cell directed against CD19 is administered to the patient, and wherein the anti-CD19 antibody is administered in combination with one or more additional pharmaceutical agent.

In one aspect, a sequential therapeutic combination is disclosed comprising an anti-CD19 antibody and a CAR-T cell directed against CD19 for use in the treatment of cancer, wherein the anti-CD19 antibody is administered to a cancer patient and wherein after said patient has relapsed or is refractory, a CAR-T cell directed against CD19 is administered to the patient, and wherein the anti-CD19 antibody is administered in combination with one or more additional pharmaceutical agent, wherein said pharmaceutical agent is a biologic or a chemotherapeutic agent or a pharmaceutically acceptable salt thereof.

In a further aspect, a sequential therapeutic combination is disclosed comprising an anti-CD19 antibody and a CAR-T cell directed against CD19 for use in the treatment of cancer, wherein the anti-CD19 antibody is administered to a cancer patient and wherein after said patient has relapsed or is refractory, a CAR-T cell directed against CD19 is administered to the patient, and wherein the anti-CD19 antibody is administered in combination with one or more additional pharmaceutical agent, wherein said pharmaceutical agent is a therapeutic antibody or antibody fragment, a nitrogen mustard, a purine analog, a thalidomide analog, a phosphoinositide 3-kinase inhibitor, a BCL-2 inhibitor, a bruton's tyrosine kinase (BTK) inhibitor or a pharmaceutically acceptable salt thereof.

In a further aspect, a sequential therapeutic combination is disclosed comprising an anti-CD19 antibody and a CAR-T cell directed against CD19 for use in the treatment of cancer, wherein the anti-CD19 antibody is administered to a cancer patient and wherein after said patient has relapsed or is refractory, a CAR-T cell directed against CD19 is administered to the patient, and wherein the anti-CD19 antibody is administered in combination with one or more additional pharmaceutical agent, wherein the pharmaceutical agent is selected from the group of rituximab, R-CHOP, cyclophosphamide, chlorambucil, uramustine, ifosfamide, melphalan, bendamustine, mercaptopurine, azathioprine, thioguanine, fludarabine, thalidomide, lenalidomide, pomalidomide, idelalisib, duvelisib, copanlisib, ibrutinib, venetoclax or a pharmaceutically acceptable salt thereof.

In a preferred aspect, a sequential therapeutic combination is disclosed comprising an anti-CD19 antibody and a CAR-T cell directed against CD19 for use in the treatment of cancer, wherein the anti-CD19 antibody is administered to a cancer patient and wherein after said patient has relapsed or is refractory, a CAR-T cell directed against CD19 is administered to the patient, and wherein the anti-CD19 antibody is administered in combination with one or more additional pharmaceutical agent, wherein the pharmaceutical agent is lenalidomide or a pharmaceutically acceptable salt thereof.

In a particular aspect, a sequential therapeutic combination is disclosed comprising an anti-CD19 antibody and a CAR-T cell directed against CD19 for use in the treatment of cancer, wherein the anti-CD19 antibody is administered to a cancer patient and wherein after said patient has relapsed or is refractory, a CAR-T cell directed against CD19 is administered to the patient, and wherein the anti-CD19 antibody is administered in combination with lenalidomide or a pharmaceutically acceptable salt thereof, and wherein said anti-CD19 antibody is administered at least bi-weekly at an amount of 12 mg/kg per dose and wherein lenalidomide is administered daily at an amount of 25 mg.

In a certain aspect, a sequential therapeutic combination is disclosed comprising an anti-CD19 antibody and a CAR-T cell directed against CD19 for use in the treatment of cancer, wherein the anti-CD19 antibody is administered to a cancer patient and wherein after said patient has relapsed or is refractory, a chimeric antigen receptor (CAR) T cell directed against CD19 is administered to the patient, wherein the anti-CD19 antibody is administered in combination with lenalidomide or a pharmaceutically acceptable salt thereof, and wherein said anti-CD19 antibody is administered at least bi-weekly at an amount of 12 mg/kg per dose and wherein lenalidomide is administered daily at an amount of 25 mg, and wherein said anti-CD19 antibody is administered in a regimen of up to 12 cycles wherein for cycles 1 to 3, said anti-CD19 antibody is administered weekly and from cycle 4 onwards, said anti-CD19 antibody is administered every 14 days and wherein lenalidomide is administered daily.

In preferred aspects, of the sequential therapeutic combinations disclosed herein, comprising an anti-CD19 antibody and a CAR-T cell directed against CD19 for use in the treatment of cancer, the anti-CD19 antibody comprises an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6).

In other aspects, of the sequential therapeutic combinations disclosed herein, comprising an anti-CD19 antibody and a CAR-T cell directed against CD19 for use in the treatment of cancer, the anti-CD19 antibody comprises a variable heavy chain of SEQ ID NO: 7 and a variable light chain of SEQ ID NO: 8. In particular aspects, the sequential therapeutic combinations disclosed herein comprise an anti-CD19 antibody and a CAR-T cell directed against CD19 for use in the treatment of cancer, the anti-CD19 antibody comprising a heavy chain of SEQ ID NO: 11 and a light chain of SEQ ID NO: 12. In some aspects, the sequential therapeutic combinations disclosed herein comprise tafasitamab and a CAR-T cell directed against CD19 for use in the treatment of cancer.

In preferred aspects, of the sequential therapeutic combinations disclosed herein, comprising an anti-CD19 antibody and a CAR-T cell directed against CD19 for use in the treatment of cancer, the anti-CD19 antibody comprises
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and a variable light chain of SEQ ID NO: 8, or
iii) a heavy chain of SEQ ID NO: 11 and a light chain of SEQ ID NO: 12, or
iv) tafasitamab,
   and the CAR-T cell directed against CD19 comprises one of or a combination of two or more of axicabtagen-ciloleucel (KTE-C19, Axi-cel, YESCARTA®) and/or tisagenlecleucel (CTL019, KYMRIAH®), lisocabtagene maraleucel (JCAR017), or UCART19 (S 68587). In another aspect, the CAR-T cell is one of or a combination of two or more of CAR-T cell constructs known as KITE037, welgenaleucel, ICTCAR-003, IM-19, CTX-110, SSCAR-010, ICTCAR-011.

In preferred aspects, of the sequential therapeutic combinations disclosed herein, comprising an anti-CD19 antibody and a CAR-T cell directed against CD19 for use in the treatment of cancer, the cancer is a hematological cancer selected from non-Hodgkin's B cell lymphoma, such as for example follicular lymphoma (FL), small lymphocytic lymphoma (SLL), Burkitt lymphoma and diffuse large B-cell lymphoma (DLBCL), Waldenstrom's macroglobulinemia, leukemia, such as for example chronic lymphocytic leukemia (CLL), acute lymphoblastic leukemia (ALL) or acute myeloid leukemia (AML). In a preferred embodiment, the hematological cancer is DLBCL. In other embodiments, the cancer is relapsed or refractory (R/R) cancer, preferably a relapsed or refractory hematological cancer. The R/R hematological cancer is selected from R/R non-Hodgkin's B cell lymphoma, such as for example R/R follicular lymphoma (FL), R/R small lymphocytic lymphoma (SLL), R/R Burkitt lymphoma and R/R diffuse large B-cell lymphoma (DLBCL), R/R Waldenstrom's macroglobulinemia, R/R leukemia, such as for example R/R chronic lymphocytic leukemia (CLL), R/R acute lymphoblastic leukemia (ALL) or R/R acute myeloid leukemia (AML). In a preferred embodiment, the R/R hematological cancer is relapsed or refractory (R/R) Diffuse Large B-Cell Lymphoma (DLBCL).

In one preferred aspect, the sequential therapeutic combination comprises tafasitamab and axicabtagen-ciloleucel (YESCARTA®) CAR-T cells for use in the treatment of DLBCL in a patient, wherein the patient is refractory or relapsed from previous treatment.

In one preferred aspect, the sequential therapeutic combination comprises tafasitamab and axicabtagen-ciloleucel (YESCARTA®) CAR-T cells for use in the treatment of DLBCL in a patient, wherein the patient is refractory or relapsed from previous treatment, the previous treatment comprising tafasitamab.

In another embodiment, an anti-CD19 antibody is disclosed for use in the treatment of cancer in a patient, wherein the patient was previously treated with a composition comprising a therapeutic agent directed against CD19. In a certain embodiment, an anti-CD19 antibody is disclosed for use in the treatment of cancer in a patient, wherein the patient was previously treated with a composition comprising a CAR-T cell directed against CD19.

In a preferred embodiment, an anti-CD19 antibody, comprising an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6) is disclosed, for use in the treatment of cancer in a patient, wherein the patient is refractory or relapsed from previous treatment, the previous treatment comprising a CAR-T cell directed against CD19.

In another embodiment, an anti-CD19 antibody comprising a variable heavy chain comprising SEQ ID NO: 7 and/or a variable light chain comprising SEQ ID NO: 8 is disclosed, for use in the treatment of cancer in a patient, wherein the patient is refractory or relapsed from previous treatment, the previous treatment comprising a CAR-T cell directed against CD19.

In one embodiment, an anti-CD19 antibody comprising a variable heavy chain and/or a variable light chain with at least 90%, at least 91%, at least 92%, at least 93%, at least 94%, at least 95%, at least 96%, at least 97%, at least 98% or at least 99% sequence identity to the variable heavy chain of SEQ ID NO: 7 and/or the variable light chain of SEQ ID NO: 8 is disclosed, for use in the treatment of cancer in a patient, wherein the patient is refractory or relapsed from previous treatment, the previous treatment comprising a CAR-T cell directed against CD19.

In a certain embodiment, an anti-CD19 antibody comprising a heavy chain of SEQ ID NO: 11 and a light chain of SEQ ID NO: 12 is disclosed, for use in the treatment of cancer in a patient, wherein the patient is refractory or relapsed from previous treatment, the previous treatment comprising a CAR-T cell directed against CD19.

In a particular embodiment, an anti-CD19 antibody comprising tafasitamab is disclosed, for use in the treatment of cancer in a patient, wherein the patient is refractory or relapsed from previous treatment, the previous treatment comprising a CAR-T cell directed against CD19.

In another embodiment, an anti-CD19 antibody is disclosed for use in the treatment of cancer in a patient, wherein the patient is refractory or relapsed from previous treatment, the previous treatment comprising anti-CD19 CAR-T cells, of which the chimeric antigen receptor is competing with the anti-CD19 antibody for binding to human CD19.

In other embodiments, an anti-CD19 antibody is disclosed for use in the treatment of cancer in a patient, wherein the patient is refractory or relapsed from previous treatment, the previous treatment comprising an anti-CD19 therapeutic agent, and wherein the therapeutic agent is selected from the group of an antibody, an antibody-drug conjugate, a bispecific, an alternative scaffold protein or a CAR-T cell.

In a preferred embodiment, an anti-CD19 antibody is disclosed for use in the treatment of cancer in a patient, wherein the patient is refractory or relapsed from previous treatment, the previous treatment comprising an anti-CD19 CAR-T cell, and wherein the anti-CD19 antibody comprises tafasitamab. In certain embodiments, the CAR-T cell is preferably one of or a combination of two or more of axicabtagen-ciloleucel (KTE-C19, Axi-cel, YESCARTA®) and/or tisagenlecleucel (CTL019, KYMRIAH®), lisocabtagene maraleucel (JCAR017, clinical trials: e.g. NCT02631044, NCT03310619), or UCART19. In another embodiment the CAR-T cell is one of or a combination of two or more of CAR-T cell constructs known as KITE037, welgenaleucel, ICTCAR-003, IM-19, CTX-110, SSCAR-010, ICTCAR-011.

In other embodiments, an antibody is directed against a B cell lineage marker for use in the treatment of cancer in a patient, wherein the patient is refractory or relapsed from previous treatment, the previous treatment comprising a therapeutic anti-CD19 agent.

In another embodiment, the antibody is directed against CD19 and/or CD20 for use in the treatment of cancer in a patient, wherein the patient is refractory or relapsed from previous treatment, the previous treatment comprising a therapeutic anti-CD19 agent.

In other embodiments, the antibody is directed against a B cell lineage marker for use in the treatment of cancer in a patient, wherein the patient is refractory or relapsed from previous treatment, the previous treatment comprising CAR-T cells directed against CD19.

In another embodiment, the antibody is directed against CD19 and/or CD20 for use in the treatment of cancer in a patient, wherein the patient is refractory or relapsed from previous treatment, the previous treatment comprising CAR-T cells directed against CD19.

In preferred aspects, said antibody directed against a B cell lineage marker (e.g. CD19 and/or CD20) is for use in a pharmaceutical composition. In other aspects, said antibody directed against a B cell lineage marker (e.g. CD19 and/or CD20) is comprised in a pharmaceutical composition.

In one embodiment, an anti-CD19 antibody is disclosed for use in the treatment of cancer in a patient, wherein the patient was previously treated with a composition comprising CAR-T cells directed against CD19 and wherein the cancer is a hematological cancer. In certain embodiments, the hematological cancer is selected from non-Hodgkin's B cell lymphoma, such as for example follicular lymphoma (FL), small lymphocytic lymphoma (SLL), Burkitt lymphoma and diffuse large B-cell lymphoma (DLBCL), Waldenstrom's macroglobulinemia, leukemia, such as for example chronic lymphocytic leukemia (CLL), acute lymphoblastic leukemia (ALL) or acute myeloid leukemia (AML). In a preferred embodiment, the hematological cancer is DLBCL. In other embodiments, the cancer is relapsed or refractory (R/R) cancer, preferably a relapsed or refractory hematological cancer. The R/R hematological cancer is selected from R/R non-Hodgkin's B cell lymphoma, such as for example R/R follicular lymphoma (FL), R/R small lymphocytic lymphoma (SLL), R/R Burkitt lymphoma and R/R diffuse large B-cell lymphoma (DLBCL), R/R Waldenstrom's macroglobulinemia, R/R leukemia, such as for example R/R chronic lymphocytic leukemia (CLL), R/R acute lymphoblastic leukemia (ALL) or R/R acute myeloid leukemia (AML). In a preferred embodiment, the R/R hematological cancer is relapsed or refractory (R/R) Diffuse Large B-Cell Lymphoma (DLBCL).

In a particular embodiment, an anti-CD19 antibody is disclosed for use in the treatment of R/R DLBCL in a patient, wherein the patient is refractory or relapsed from previous treatment, the previous treatment comprising CAR-T cells directed against CD19, wherein the anti-CD19 antibody comprises
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab.

In a particular embodiment, tafasitamab in combination with lenalidomide is disclosed for use in the treatment of R/R DLBCL in a patient, wherein the patient is refractory or relapsed from previous treatment, the previous treatment comprising CAR-T cells directed against CD19.

In another embodiment, tafasitamab in combination with bendamustine is disclosed for use in the treatment of R/R DLBCL in a patient, wherein the patient is refractory or relapsed from previous treatment, the previous treatment comprising CAR-T cells directed against CD19.

### Method of Treatment

In one embodiment, a method is disclosed for treating cancer in a patient with CAR-T cells directed against CD19, wherein the patient is refractory or relapsed from previous treatment, the previous treatment comprising an anti-CD19 antibody.

In one embodiment, a method is disclosed for treating cancer in a patient with a pharmaceutical composition comprising CAR-T cells directed against CD19, wherein the patient is refractory or relapsed from previous treatment, the previous treatment comprising an anti-CD19 antibody.

In one embodiment, a method is disclosed for treating cancer in a patient with a pharmaceutical composition comprising CAR-T cells directed against CD19 in a second-, third-, fourth- or any other multi-line treatment, wherein the patient is refractory or relapsed from the prior line of treatment, the prior line comprising an anti-CD19 antibody.

In another embodiment, a method is disclosed for treating cancer in a patient with a pharmaceutical composition comprising CAR-T cells directed against CD19 in a second-, third-, fourth- or any other multi-line treatment, wherein the patient received in at least one of the prior line(s) of treatment a pharmaceutical composition comprising an anti-CD19 antibody.

In another embodiment, a method is disclosed for treating cancer in a patient with a pharmaceutical composition comprising CAR-T cells directed against CD19 in a second-, third-, fourth- or any other multi-line treatment, wherein the patient received in at least one of the prior line(s) of treatment a pharmaceutical composition comprising an anti-CD19 antibody and wherein the patient is refractory or relapsed after at least one of the prior line(s) of treatment.

In another embodiment, a method is disclosed for treating a hematological cancer in a patient with a pharmaceutical composition comprising CAR-T cells directed against CD19 in a second-, third-, fourth- or any other multi-line treatment, wherein the patient received in at least one of the prior line(s) of treatment a pharmaceutical composition comprising tafasitamab.

In one embodiment, a method is disclosed for treating cancer in a patient in a second-, third-, fourth- or any other multi-line treatment comprising the steps of:
i) identifying a patient who has received a pharmaceutical composition comprising an anti-CD19 antibody, the antibody comprising an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), and
ii) administering to the patient in said second-, third-, fourth- or any other multi-line treatment a further pharmaceutical composition comprising a therapeutic agent directed against CD19.

In one embodiment, a method is disclosed for treating cancer in a patient in a second-, third-, fourth- or any other multi-line treatment comprising the steps of:
i) identifying a patient who has received a pharmaceutical composition comprising an anti-CD19 antibody, the antibody comprising a variable heavy chain of SEQ ID NO: 7 and/or a variable light chain of SEQ ID NO: 8, and
ii) administering to the patient in said second-, third-, fourth- or any other multi-line treatment a further pharmaceutical composition comprising a therapeutic agent directed against CD19.

In one embodiment, a method is disclosed for treating cancer in a patient in a second-, third-, fourth- or any other multi-line treatment comprising the steps of:
i) identifying a patient who has received a pharmaceutical composition comprising an anti-CD19 antibody, the antibody comprising a heavy chain of SEQ ID NO: 11 and/or a light chain of SEQ ID NO: 12, and
ii) administering to the patient in said second-, third-, fourth- or any other multi-line treatment a further pharmaceutical composition comprising a therapeutic agent directed against CD19.

In one embodiment, a method is disclosed for treating cancer in a patient in a second-, third-, fourth- or any other multi-line treatment comprising the steps of:
i) identifying a patient who has received a pharmaceutical composition comprising the anti-CD19 antibody tafasitamab, and
ii) administering to the patient in said second-, third-, fourth- or any other multi-line treatment a further pharmaceutical composition comprising a therapeutic agent directed against CD19.

In certain embodiments, the therapeutic agent directed against CD19 of the second-, third-, fourth- or any other multi-line therapy in step ii) is a monoclonal antibody, an antibody drug conjugate, a bispecific, an alternative scaffold protein or a chimeric antigen receptor (CAR) T cell. Preferably, the therapeutic agent directed against CD19 of the pharmaceutical composition of the second-, third-, fourth- or any other multi-line therapy is a CAR-T cell.

In one embodiment, a method is disclosed for treating cancer in a patient in a second-, third-, fourth- or any other multi-line treatment comprising the steps of:
i) identifying a patient who has received a pharmaceutical composition comprising the anti-CD19 antibody tafasitamab, and
ii) administering to the patient in said second-, third-, fourth- or any other multi-line treatment a further pharmaceutical composition comprising CAR-T cells directed against CD19.

In one embodiment, a method is disclosed for treating cancer in a patient in a second-, third-, fourth- or any other multi-line treatment comprising the steps of:
i) identifying a patient who has received a pharmaceutical composition comprising the anti-CD19 antibody tafasitamab, and
ii) administering to the patient in said second-, third-, fourth- or any other multi-line treatment a further pharmaceutical composition comprising CAR-T cells directed against CD19, wherein the CAR-T cells are selected from one or a combination of axicabtagen-ciloleucel, tisagenlecleucel, lisocabtagene maraleucel and/or UCART19.

In one embodiment, a method is disclosed for treating R/R DLBCL in a patient in a second-, third-, fourth- or any other multi-line treatment comprising the steps of:
i) identifying a patient who has received a pharmaceutical composition comprising the anti-CD19 antibody tafasitamab, and
ii) administering to the patient in said second-, third-, fourth- or any other multi-line treatment a further pharmaceutical composition comprising CAR-T cells directed against CD19, wherein the CAR-T cells are selected from one or a combination of axicabtagen-ciloleucel, tisagenlecleucel, lisocabtagene maraleucel and/or UCART19.

In some embodiments, a method of treating a relapsed or refractory CD19+ hematological cancer with a therapeutic anti-CD19 agent is disclosed, comprising the steps of
a) identifying a patient with relapsed or refractory CD19+ hematological cancer and
b) administering a therapeutic agent directed against CD19 in an effective amount to said patient,
   wherein the patient has been treated prior to step a) with a pharmaceutical composition comprising an anti-CD19 antibody, the antibody comprising
   i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
   ii) a variable heavy chain of SEQ ID NO: 7 and variable light chain of SEQ ID NO: 8, or
   iii) a heavy chain of SEQ ID NO: 11 and a light chain of SEQ ID NO: 12, or
   iv) tafasitamab.

In particular embodiments, a method of treating relapsed or refractory CD19+ hematological cancer with CAR-T cells directed against CD19 is disclosed, comprising the steps of
a) identifying a patient with relapsed or refractory CD19+ hematological cancer and
b) administering the CAR-T cells directed against CD19 in an effective amount to said patient, wherein the patient has been treated prior to step a) with a pharmaceutical composition comprising an anti-CD19 antibody, the antibody comprising
   i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
   ii) a variable heavy chain of SEQ ID NO: 7 and variable light chain of SEQ ID NO: 8, or
   iii) a heavy chain of SEQ ID NO: 11 and a light chain of SEQ ID NO: 12, or
   iv) tafasitamab.

In particular embodiments, a method of treating relapsed or refractory DLBCL with CAR-T cells directed against CD19 is disclosed, comprising the steps of
a) identifying a patient with relapsed or refractory DLBCL and
b) administering the CAR-T cells directed against CD19 in an effective amount to said patient, wherein the patient has been treated prior to step a) with a pharmaceutical composition comprising an anti-CD19 antibody, the antibody comprising
   i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
   ii) a variable heavy chain of SEQ ID NO: 7 and variable light chain of SEQ ID NO: 8, or
   iii) a heavy chain of SEQ ID NO: 11 and a light chain of SEQ ID NO: 12, or
   iv) tafasitamab.

Provided in some aspects are methods of treatment that involve: (1) administering, to a patient having a cancer, a composition comprising an anti-CD19 antibody, the antibody comprising
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab,
   and (2) administering subsequently to the patient, wherein the patient is relapsed or refractory, a composition comprising T cells that are autologous to the patient and express a recombinant receptor that specifically binds to CD19.

In a preferred embodiment, the autologous T cell directed against CD19 for use in the treatment of cancer in a patient, wherein the patient was previously treated with a composition comprising an anti-CD19 antibody, is a chimeric antigen receptor (CAR) T cell. In certain embodiments, the CAR-T cell is preferably one of or a combination of two or more of axicabtagen-ciloleucel (KTE-C19, Axi-cel, YESCARTAO) and/or tisagenlecleucel (CTL019, KYMRIAH®), lisocabtagene maraleucel (JCAR017, clinical trials: e.g. NCT02631044, NCT03310619), or UCART19. In another embodiment the CAR-T cell is one of or a combination of two or more of CAR-T cell constructs known as KITE037, welgenaleucel, ICTCAR-003, IM-19, CTX-110, SSCAR-010, ICTCAR-011.

In one embodiment, a method is disclosed for treating cancer in a patient with an anti-CD19 antibody, wherein the patient is refractory or relapsed from previous treatment, the previous treatment comprising CAR-T cells directed against CD19.

In one embodiment, a method is disclosed for treating cancer in a patient with a composition comprising an anti-CD19 antibody, wherein the patient is refractory or relapsed from previous treatment, the previous treatment comprising CAR-T cells directed against CD19.

In one embodiment, a method is disclosed for treating cancer in a patient with a pharmaceutical composition comprising an anti-CD19 antibody in a second-, third-, fourth- or any other multi-line treatment, wherein the patient is refractory or relapsed and received in the prior line of treatment a pharmaceutical composition comprising CAR-T cells directed against CD19.

In another embodiment, a method is disclosed for treating cancer in a patient with a pharmaceutical composition comprising an anti-CD19 antibody in a second-, third-, fourth- or any other multi-line treatment, wherein the patient is refractory or relapsed and received in one of the prior line(s) of treatment a pharmaceutical composition comprising CAR-T cells directed against CD19.

In one embodiment, a method is disclosed for treating cancer in a patient in a second-, third-, fourth- or any other multi-line treatment comprising the steps of:
i) identifying a patient who has received a pharmaceutical composition comprising CAR-T cells directed against CD19, and
ii) administering to the patient in said second-, third-, fourth- or any other multi-line treatment a further pharmaceutical composition comprising an anti-CD19 antibody, the antibody comprising an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6).

In one embodiment, a method is disclosed for treating cancer in a patient in a second-, third-, fourth- or any other multi-line treatment comprising the steps of:
i) identifying a patient who has received a pharmaceutical composition comprising CAR-T cells directed against CD19, and
ii) administering to the patient in said second-, third-, fourth- or any other multi-line treatment a further pharmaceutical composition comprising an anti-CD19 antibody, the antibody comprising a variable heavy chain of SEQ ID NO: 7 and/or a variable light chain of SEQ ID NO: 8.

In one embodiment, a method is disclosed for treating cancer in a patient in a second-, third-, fourth- or any other multi-line treatment comprising the steps of:
i) identifying a patient who has received a pharmaceutical composition comprising CAR-T cells directed against CD19, and
ii) administering to the patient in said second-, third-, fourth- or any other multi-line treatment a further pharmaceutical composition comprising an anti-CD19 antibody, the antibody comprising a heavy chain of SEQ ID NO: 11 and/or a light chain of SEQ ID NO: 12.

In one embodiment, a method is disclosed for treating cancer in a patient in a second-, third-, fourth- or any other multi-line treatment comprising the steps of:
i) identifying a patient who has received a pharmaceutical composition comprising CAR-T cells directed against CD19, and
ii) administering to the patient in said second-, third-, fourth- or any other multi-line treatment a further pharmaceutical composition comprising the anti-CD19 antibody tafasitamab.

In one embodiment, a method is disclosed for treating cancer in a patient in a second-, third-, fourth- or any other multi-line treatment comprising the steps of:
i) identifying a patient who has received a pharmaceutical composition comprising CAR-T cells directed against CD19, wherein the CAR-T cells are selected from one or a combination of axicabtagen-ciloleucel, tisagenlecleucel, lisocabtagene maraleucel and/or UCART19, and
ii) administering to the patient in said second-, third-, fourth- or any other multi-line treatment a further pharmaceutical composition comprising the anti-CD19 antibody tafasitamab.

In one embodiment, a method is disclosed for treating R/R DLBCL in a patient in a second-, third-, fourth- or any other multi-line treatment comprising the steps of:
i) identifying a patient who has received a pharmaceutical composition comprising CAR-T cells directed against CD19, wherein the CAR-T cells are selected from one or a combination of axicabtagen-ciloleucel, tisagenlecleucel, lisocabtagene maraleucel and/or UCART19, and
ii) administering to the patient in said second-, third-, fourth- or any other multi-line treatment a further pharmaceutical composition comprising the anti-CD19 antibody tafasitamab.

In some embodiments, a method of treating relapsed or refractory CD19+ hematological cancer with an anti-CD19 antibody is disclosed, comprising the steps of
a) identifying a patient with relapsed or refractory CD19+ hematological cancer and
b) administering the anti-CD19 antibody in an effective amount to said patient, wherein the patient has been treated prior to step a) with a pharmaceutical composition comprising CAR-T cells directed against CD19, and wherein the anti-CD19 antibody in step b) comprises
   i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
   ii) a variable heavy chain of SEQ ID NO: 7 and variable light chain of SEQ ID NO: 8, or
   iii) a heavy chain of SEQ ID NO: 11 and a light chain of SEQ ID NO: 12, or
   iv) tafasitamab.

Provided in some aspects are methods of treatment that involve: (1) administering, to a patient having a cancer, a composition comprising T cells that are autologous to the patient and express a recombinant receptor that specifically binds to CD19 associated with the cancer and (2) administering subsequently to the patient, wherein the patient is refractory or relapsed, a composition comprising an anti-CD19 antibody, the antibody comprising
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab.

In a preferred embodiment, the autologous T cells that are directed against CD19 for use in the treatment of cancer in a patient, wherein the patient is refractory or relapsed and wherein the patient is subsequently treated with a composition comprising an anti-CD19 antibody, is a chimeric antigen receptor (CAR) T cell. In certain embodiments, the CAR-T cell is preferably one of or a combination of two or more of axicabtagen-ciloleucel (KTE-C19, Axi-cel, YESCARTA®) and/or tisagenlecleucel (CTL019, KYMRIAHO), lisocabtagene maraleucel (JCAR017, clinical trials: e.g. NCT02631044, NCT03310619), or UCART19. In another embodiment the CAR-T cell is one of or a combination of two or more of (CAR) T cell constructs known as KITE037, welgenaleucel, ICTCAR-003, IM-19, CTX-110, SSCAR-010, ICTCAR-011.

### Method of Predicting Response

In another embodiment, a method for determining if a patient is likely to respond or is not likely to respond to CAR-T cell therapy directed against CD19, subsequent to treatment with tafasitamab is disclosed, comprising the steps of
a) contacting a tumor sample isolated from the patient with an effective amount of an anti-CD19 antibody comprising a variable heavy chain of SEQ ID NO: 7 and a variable light chain of SEQ ID NO: 8 to saturate the antigen in vitro,
b) adding CAR-T cells directed against CD19 at different effector : target ratios, and
c) assessing CAR-T cell effector function (e.g.: antigen specific killing, degranulation, cytokine production or proliferation),
wherein the presence of CAR-T cell effector function indicates that the patient is likely to respond to anti-CD19 CAR-T cell therapy and the absence of CAR-T cell effector function indicates that the patient is not likely to respond to the anti-CD19 CAR-T cell therapy.

In another embodiment, a method for determining if a patient, who is refractory or relapsed, is likely to respond or is not likely to respond to CAR-T cell therapy directed against CD19, subsequent to treatment with tafasitamab is disclosed, comprising the steps of
a) contacting a tumor sample isolated from the patient with an effective amount of an anti-CD19 antibody comprising a variable heavy chain of SEQ ID NO: 7 and a variable light chain of SEQ ID NO: 8 to saturate the antigen in vitro,
b) adding CAR-T cells directed against CD19 at different effector:target ratios, and
c) assessing CAR-T cell effector function (e.g.: antigen specific killing, degranulation, cytokine production or proliferation),
wherein the presence of CAR-T cell effector function indicates that the patient is likely to respond to anti-CD19 CAR-T cell therapy and the absence of CAR-T cell effector function indicates that the patient is not likely to respond to the anti-CD19 CAR-T cell therapy.

In particular embodiments, the different effector:target (E:T) ratios are 0:1, 0.3125:1, 0.625:1, 1.25:1, 2.5:1, 5:1 and 10:1. In one embodiment the anti-CD19 CAR-T cell comprises a CAR construct of the format FMC63-CD8h-CD8TM-41BEK.

Use of a therapeutic agent directed against CD19 in the manufacture of a medicament for the treatment of a hematological cancer in a patient, wherein the patient is refractory or relapsed from previous treatment, the previous treatment comprising an anti-CD19 antibody, the antibody comprising
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab.

Use of CAR-T cells directed against CD19 in the manufacture of a medicament for the treatment of a hematological cancer in a patient, wherein the patient is refractory or relapsed from previous treatment, the previous treatment comprising an anti-CD19 antibody, the antibody comprising
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab.

Use of CAR-T cells directed against CD19 in the manufacture of a medicament for the treatment of a hematological cancer in a patient, wherein the patient is refractory or relapsed from previous treatment, the previous treatment comprising an anti-CD19 antibody, the antibody comprising
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab,
   and wherein the CAR-T cells directed against CD19 are selected from the group of axicabtagen-ciloleucel (YESCARTA®), tisagenlecleucel (KYMRIAH®), lisocabtagene maraleucel , or UCART19.

Use of CAR-T cells directed against CD19 in the manufacture of a medicament for the treatment of a hematological cancer in a patient, wherein the patient is refractory or relapsed from previous treatment, the previous treatment comprising an anti-CD19 antibody, the antibody comprising
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab,
   and wherein the hematological cancer is selected from non-Hodgkin's B cell lymphoma, such as for example follicular lymphoma (FL), small lymphocytic lymphoma (SLL), Burkitt lymphoma and diffuse large B-cell lymphoma (DLBCL), Waldenstrom's macroglobulinemia, leukemia, such as for example chronic lymphocytic leukemia (CLL), acute lymphoblastic leukemia (ALL) or acute myeloid leukemia (AML), relapsed or refractory (R/R) non-Hodgkin's B cell lymphoma, such as for example R/R follicular lymphoma (FL), R/R small lymphocytic lymphoma (SLL), R/R Burkitt lymphoma and R/R diffuse large B-cell lymphoma (DLBCL), R/R Waldenstrom's macroglobulinemia, R/R leukemia, such as for example R/R chronic lymphocytic leukemia (CLL), R/R acute lymphoblastic leukemia (ALL) or R/R acute myeloid leukemia (AML), preferably R/R DLBCL.

Use of axicabtagen-ciloleucel (YESCARTA®) CAR-T cells directed against CD19 in the manufacture of a medicament for the treatment of R/R DLBCL in a patient, wherein the patient is refractory or relapsed from previous treatment, the previous treatment comprising the anti-CD19 antibody tafasitamab.

Use of an anti-CD19 antibody in the manufacture of a medicament for the treatment of a hematological cancer in a patient, wherein the patient is refractory or relapsed from previous treatment, the previous treatment comprising CAR-T cells directed against CD19, and wherein the anti-CD19 antibody comprises
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab.

Use of an anti-CD19 antibody in the manufacture of a medicament for the treatment of a hematological cancer in a patient, wherein the patient is refractory or relapsed from previous treatment, the previous treatment comprising CAR-T cells directed against CD19, and wherein the anti-C19 antibody comprises
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab,
   and wherein the CAR-T cells directed against CD19 are selected from the group of axicabtagen-ciloleucel (YESCARTA®), tisagenlecleucel (KYMRIAH®), lisocabtagene maraleucel, or UCART19.

Use of an anti-CD19 antibody in the manufacture of a medicament for the treatment of a hematological cancer in a patient, wherein the patient is refractory or relapsed from previous treatment, the previous treatment comprising CAR-T cells directed against CD19, and wherein the antibody comprises
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab,
   and wherein the hematological cancer is selected from non-Hodgkin's B cell lymphoma, such as for example follicular lymphoma (FL), small lymphocytic lymphoma (SLL), Burkitt lymphoma and diffuse large B-cell lymphoma (DLBCL), Waldenstrom's macroglobulinemia, leukemia, such as for example chronic lymphocytic leukemia (CLL), acute lymphoblastic leukemia (ALL) or acute myeloid leukemia (AML), relapsed or refractory (R/R) non-Hodgkin's B cell lymphoma, such as for example R/R follicular lymphoma (FL), R/R small lymphocytic lymphoma (SLL), R/R Burkitt lymphoma and R/R diffuse large B-cell lymphoma (DLBCL), R/R Waldenstrom's macroglobulinemia, R/R leukemia, such as for example R/R chronic lymphocytic leukemia (CLL), R/R acute lymphoblastic leukemia (ALL) or R/R acute myeloid leukemia (AML), preferably R/R DLBCL.

Use of an anti-CD19 antibody in the manufacture of a medicament for the treatment of R/R DLBCL in a patient, wherein the patient is refractory or relapsed from previous treatment, the previous treatment comprising CAR-T cells directed against CD19, and wherein the antibody comprises
i) an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6), or
ii) a variable heavy chain of SEQ ID NO: 7 and variable light chain of SEQ ID NO: 8, or
iii) a heavy chain of SEQ ID NO: 11 and a light chain of SEQ ID NO: 12, or
iv) tafasitamab

Use of tafasitamab in the manufacture of a medicament for the treatment of R/R DLBCL in a patient, wherein the patient is refractory or relapsed from previous treatment, the previous treatment comprising CAR-T cells directed against CD19.

1. A therapeutic agent directed against CD19 for use in the treatment of cancer in a patient, wherein the patient is refractory or relapsed from previous treatment, the previous treatment comprising an anti-CD19 antibody comprising an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6).

2. A therapeutic agent directed against CD19 for use according to any of the preceding claims, wherein the therapeutic agent is selected from the group of an antibody, an antibody-drug conjugate, a bispecific, an alternative scaffold protein or a chimeric antigen receptor (CAR) T cell.

3. A therapeutic agent directed against CD19 for use according to claim 2, wherein the therapeutic agent is a chimeric antigen receptor (CAR) T cell.

4. A chimeric antigen receptor (CAR) T cell directed against CD19 for use according to claim 3, wherein the CAR-T cell is axicabtagene ciloleucel, tisagenlecleucel, lisocabtagene maraleucel, or UCART19.

5. A therapeutic agent directed against CD19 for use according to any of the preceding claims, wherein the cancer is hematological cancer and selected from non-Hodgkin's B cell lymphoma, follicular lymphoma (FL), small lymphocytic lymphoma (SLL), Burkitt lymphoma, diffuse large B-cell lymphoma (DLBCL), Waldenstrom's macroglobulinemia, B cell leukemia, chronic lymphocytic leukemia (CLL), or acute lymphoblastic leukemia (ALL).

6. A therapeutic agent directed against CD19 for use according to claim 5, wherein the hematological cancer is relapsed or refractory hematological cancer.

7. A therapeutic agent directed against CD19 for use according to claim 6, wherein the relapsed or refractory hematological cancer is R/R—DLBCL.

8. An anti-CD19 antibody for use in the treatment of cancer in a patient, wherein said patient after such anti-CD19 antibody treatment is treated with a chimeric antigen receptor (CAR) T cell directed against CD19 and wherein the anti-CD19 antibody comprises an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6).

9. A sequential therapeutic combination comprising an anti-CD19 antibody and a chimeric antigen receptor (CAR) T cell directed against CD19 for use in the treatment of cancer.

10. The sequential therapeutic combination according to claim 9 for use in the treatment of cancer wherein the anti-CD19 antibody is administered to a cancer patient and wherein after said patient has relapsed or is refractory, a CAR-T cell directed against CD19 is administered to the patient.

11. The sequential therapeutic combination according to claim 10 for use in the treatment of cancer wherein the anti-CD19 antibody is administered in combination with one or more additional pharmaceutical agent.

12. The sequential therapeutic combination according to claim 11 for use in the treatment of cancer wherein said pharmaceutical agent is a therapeutic antibody or antibody fragment, a nitrogen mustard, a purine analog, a thalidomide analog, a phosphoinositide 3-kinase inhibitor, a BCL-2 inhibitor, a bruton's tyrosine kinase (BTK) inhibitor or a pharmaceutically acceptable salt thereof.

13. The sequential therapeutic combination according to claim 12 for use in the treatment of cancer, wherein the pharmaceutical agent is lenalidomide or a pharmaceutically acceptable salt thereof.

14. The sequential therapeutic combination according to claim 13 for use in the treatment of cancer, wherein said anti-CD19 antibody is administered in a regimen of up to 12 cycles wherein for cycles 1 to 3, said anti-CD19 antibody is administered weekly and from cycle 4 onwards, said anti-CD19 antibody is administered every 14 days and wherein lenalidomide is administered daily.

15. A sequential therapeutic combination according to any one of the claims 9 to 14 wherein the anti-CD19 antibody comprises an HCDR1 region comprising sequence SYVMH (SEQ ID NO: 1), an HCDR2 region comprising sequence NPYNDG (SEQ ID NO: 2), an HCDR3 region comprising sequence GTYYYGTRVFDY (SEQ ID NO: 3), an LCDR1 region comprising sequence RSSKSLQNVNGNTYLY (SEQ ID NO: 4), an LCDR2 region comprising sequence RMSNLNS (SEQ ID NO: 5), and an LCDR3 region comprising sequence MQHLEYPIT (SEQ ID NO: 6).
